(19) United States
(12) Patent Application Publication
(10) Pub. No.: US 2025/0107976 A1
(43) Pub. Date: Apr. 3, 2025

(54) COMPOSITIONS AND METHODS FOR TREATING HAIR

(71) Applicant: L'Oreal, Paris (FR)

(72) Inventors: Taylor HART, New York, NY (US); Mohamed Amer ALKAHWAJI, Hoboken, NJ (US); Michael DEGEORGE, Brick, NJ (US); Emma HOWES, Lake Hiawatha, NJ (US)

(21) Appl. No.: 18/898,187

(22) Filed: Sep. 26, 2024

Related U.S. Application Data
(60) Provisional application No. 63/541,847, filed on Sep. 30, 2023.

(30) Foreign Application Priority Data
Apr. 17, 2024 (FR) 2403981

(51) Int. Cl. A61K 8/22; A61Q 5/12

(52) U.S. Cl. CPC A61K 8/22; A61Q 5/12; A61K 2800/52; A61K 2800/592

(57) ABSTRACT

The disclosure relates to hair treatment compositions comprising hydrogen peroxide for treating hair, such as for imparting shine and/or conditioning benefits to hair. The disclosure also relates to methods of treating hair with the compositions.

### TECHNICAL FIELD

The disclosure relates to compositions and methods for treating hair, in particular to impart cosmetic benefits such as conditioning and shine to hair.

### BACKGROUND

It is known that consumers desire to use cosmetic and care compositions that enhance the appearance of keratinous substrates such as hair, e.g., by changing the color, style, and/or shape of the hair, and/or by imparting various properties to hair such as shine and conditioning. Many of the known compositions and processes for enhancing the appearance of the hair involve chemical treatment of the hair. For example, the process of changing the color of hair can involve imparting an artificial color to the hair which provides a different shade or color, and/or lifting the color of the hair, such as lightening the color of dark hair to a lighter shade.

In general, hair lightening or hair dyeing compositions are prepared by mixing a hair lightening or hair dye base composition with an oxidizing composition, also known as a developer composition, just before use. Hydrogen peroxide is the most common oxidizing agent used in processes for permanently dyeing and lightening hair. However, once the developer and hair dye base or hair lightening composition are mixed, the oxidation reaction will begin substantially immediately. As such, it is known that the step of mixing the compositions should not occur until the hair to be colored or lightened is ready for the mixture to be applied. In addition, once the application process is complete, if additional mixture remains it cannot be stored for later use and must be discarded.

While hair dyeing or lightening compositions can effectively alter the color of hair, these chemical processes also damage hair fibers. Chemical processes for changing the shape of hair such as straightening or permanent waving likewise damage hair fibers. Such damage may appear as split ends, broken hair, flyaway hair, etc., and may cause the hair to feel rough and look dull and unhealthy. Therefore, consumers desire treatment compositions that can impart cosmetic benefits to hair, for example by minimizing the appearance of spit ends, imparting shine and/or softness to the hair, etc.

It has now been discovered, surprisingly, that hydrogen peroxide can be used to impart advantageous cosmetic benefits to hair. It has also been discovered that a synergistic combination of components provides unexpected stability to hair treatment compositions containing hydrogen peroxide. The disclosure thus relates to hair treatment compositions and methods for treating the hair in order to provide cosmetic advantages such as shine, conditioning, fiber strength, and/or a healthy appearance to the hair, while avoiding or minimizing damage to the hair, such as in connection with chemical processes for enhancing the appearance of the hair.

### SUMMARY

The disclosure relates to compositions and methods for treating hair. The compositions are acidic and comprise hydrogen peroxide and one or more additional components such as surfactants, silicones, fatty compounds other than silicones, polymers, amino acids, vitamins, pH adjusters, solvents, etc. The methods comprise applying the compositions according to the disclosure to the hair, which surprisingly improve the shine, conditioning, fiber strength, and/or appearance of the treated hair.

In various embodiments, the disclosure relates to hair treatment compositions comprising (a) hydrogen peroxide or a hydrogen peroxide generating agent, (b) optionally at least one surfactant, (c) optionally at least one silicone compound, (d) optionally at least one non-silicone fatty compound, (e) optionally at least one polymer, (f) optionally at least one pigment and/or direct dye, and (g) at least one solvent. In other embodiments, the disclosure relates to hair treatment compositions comprising (a) hydrogen peroxide or a hydrogen peroxide generating agent, (b) at least one surfactant, (c) optionally at least one silicone compound, (d) optionally at least one non-silicone fatty compound, (e) optionally at least one polymer, (f) optionally at least one pigment and/or direct dye, and (g) at least one solvent. In still further embodiments, the disclosure relates to hair treatment compositions comprising (a) hydrogen peroxide or a hydrogen peroxide generating agent, (b) at least one surfactant, (c) at least one silicone compound, (d) optionally at least one non-silicone fatty compound, (e) optionally at least one polymer, (f) optionally at least one pigment and/or direct dye, and (g) at least one solvent.

In one preferred embodiment, the hair treatment compositions comprise (a) hydrogen peroxide or a hydrogen peroxide generating agent, (b) at least one surfactant chosen from anionic and/or nonionic surfactants, (c) at least one silicone compound, (d) at least one non-silicone fatty compound, (e) at least one polymer, (f) optionally at least one pigment and/or direct dye, and (g) at least one solvent. In further preferred embodiments, the hair treatment compositions comprise (a) hydrogen peroxide or a hydrogen peroxide generating agent, (b1) at least one anionic surfactant, (b2) at least one nonionic surfactant, (c) at least one silicone compound, (d) at least one non-silicone fatty compound, (e) at least one polymer, (f) optionally at least one pigment and/or direct dye, and (g) at least one solvent, e.g. water and/or one or more non-aqueous solvents. In preferred embodiments, the nonionic surfactants may be chosen from sorbitol esters of C8-C24 fatty acids or alkoxylated derivatives thereof, ethoxylated fatty alcohols, or fatty acid alkanolamides, and/or the anionic surfactants may be chosen from sodium olefin sulfonates. The compositions may optionally further comprise at least one amphoteric surfactant and/or at least one cationic surfactant. In preferred embodiments, the silicone compounds may be chosen from dimethicone, amodimethicone, and/or derivatives thereof, and/or the non-silicone fatty compounds may be chosen from plant oils. In preferred embodiments, the polymers may be chosen from carbomers, quaternary ammonium polymers, and/or polysaccharide polymers. The hair treatment compositions may optionally comprise further components, for example amino acids and/or amino sulfonic acids, hydrogen peroxide stabilizers, and/or antioxidants.

In preferred embodiments, the hair treatment compositions comprise (a) greater than about 0.5%, preferably from about 1% to about 10%, more preferably from about 1% to about 8%, most preferably from about 1% to about 6% hydrogen peroxide or a hydrogen peroxide generating agent, (b1) from about 0.01% to about 10%, preferably from about 0.1% to about 8%, more preferably from about 0.5% to about 6%, most preferably from about 1% to about 5% of anionic surfactants, (b2) from about 0.01% to about 10%, preferably from about 0.1% to about 8%, more preferably from about 0.5% to about 6%, most preferably from about 1% to about 5% of nonionic surfactants, (c) from about 0.1% to about 8%, preferably from about 0.5% to about 6%, more preferably from about 1% to about 5% of silicone compounds, preferably chosen from dimethicone, amodimethicone, and/or derivatives thereof, (d) from about 0.1% to about 8%, preferably from about 0.5% to about 6%, more preferably from about 0.5% to about 4% of non-silicone fatty compounds, preferably comprising at least one plant oil, (e) from about 0.01% to about 5%, preferably from about 0.05% to about 4%, more preferably from about 0.1% to about 3%, most preferably from about 0.2% to about 2% of polymers, preferably chosen from carbomers, (f) at least one solvent, preferably comprising water and at least one non-aqueous solvent, and (g) optionally at least one additional component chosen from pigments and/or direct dyes, amino acids and/or amino sulfonic acids, hydrogen peroxide stabilizers, cationic and/or amphoteric surfactants, pH adjusters, and/or antioxidants, where all amounts are by weight, relative to the total weight of the hair treatment composition. The pH of the hair treatment composition is less than about 7, preferably ranging from about 2.5 to about 6.5, more preferably from about 3 to about 5, most preferably from about 3.5 to about 4.5.

In further preferred embodiments, the hair treatment compositions comprise (a) from about (or exactly) 1.5% to about (or exactly) 8%, preferably from about (or exactly) 1.5% to about 6% of hydrogen peroxide, (b1) from about 0.01% to about 10%, preferably from about 0.1% to about 8%, more preferably from about 0.5% to about 6%, most preferably from about 1% to about 5% of anionic surfactants, preferably chosen from non-sulfate anionic surfactants, e.g. sodium olefin sulfonates, (b2) from about 0.01% to about 10%, preferably from about 0.1% to about 8%, more preferably from about 0.5% to about 6%, most preferably from about 1% to about 5% of nonionic surfactants, preferably chosen from sorbitol esters of C8-C24 fatty acids or alkoxylated derivatives thereof, ethoxylated fatty alcohols, or fatty acid alkanolamides, (c) from about 0.1% to about 8%, preferably from about 0.5% to about 6%, more preferably from about 1% to about 5% of silicone compounds, preferably chosen from dimethicone, amodimethicone, and/or derivatives thereof, (d) from about 0.1% to about 5%, preferably from about 0.5% to about 4%, more preferably from about 0.5% to about 3% of plant oils, (e) from about 0.01% to about 5%, preferably from about 0.05% to about 4%, more preferably from about 0.1% to about 3%, most preferably from about 0.2% to about 2% of carbomers, (f) at least one solvent, preferably comprising water and at least one non-aqueous solvent, and (g) optionally at least one additional component chosen from pigments and/or direct dyes, amino acids and/or amino sulfonic acids, hydrogen peroxide stabilizers, cationic and/or amphoteric surfactants, pH adjusters, and/or antioxidants, where all amounts are by weight, relative to the total weight of the hair treatment composition. The pH of the hair treatment composition is less than about 7, preferably ranging from about 2.5 to about 6.5, more preferably from about 3 to about 5, most preferably from about 3.5 to about 4.5.

In still further preferred embodiments, the hair treatment compositions comprise (a) from no less than 1.5% to about 6% of hydrogen peroxide, (b1) from about 1% to about 5% of anionic surfactants chosen from anionic surfactants other than sulfate surfactants, (b2) from about 1% to about 5% of nonionic surfactants, (c) from about 1% to about 5% of silicone compounds chosen from dimethicone, amodimethicone, and/or derivatives thereof, (d) from about 0.5% to about 2% of plant oils, (e) from about 0.2% to about 2% of carbomers, (f) water and optionally at least one non-aqueous solvent, and (g) at least one additional component chosen from pigments and/or direct dyes, amino acids and/or amino sulfonic acids, hydrogen peroxide stabilizers, cationic and/or amphoteric surfactants, pH adjusters, and/or antioxidants, where all amounts are by weight, relative to the total weight of the hair treatment composition. The pH of the hair treatment composition is less than about 7, preferably ranging from about 2.5 to about 6.5, more preferably from about 3 to about 5, most preferably from about 3.5 to about 4.5.

Hair treatment compositions according to the disclosure are surprisingly stable despite the presence of hydrogen peroxide. For example, the compositions may be stable for at least 24 hours, such as at least 48 hours, at least 72 hours, at least one week, at least two weeks, at least one month, at least two months, or even at least six months.

The disclosure also relates to methods of treating hair with the hair treatment compositions. The methods comprise applying a hair treatment composition as described to hair that is dry, damp, or wet, and leaving the composition on the hair for a leave-in time ranging from at least about 30 seconds, such as at least about 1 minute to less than about 20 minutes, for example less than about 15 minutes, less than about 12 minutes, or less than about 10 minutes. The composition may then be removed from the hair, for example by rinsing the hair. The methods surprisingly impart cosmetic benefits to the treated hair such as conditioning, frizz control, ease of detangling or combing, strength, shine, and/or non-permanent color.

The disclosure also relates to kits, where the kits comprise at least one, such as at least two, compartments or containers. In some embodiments, a first compartment or container comprises hydrogen peroxide and/or a hydrogen peroxide generating agent, and a second compartment or container comprises a composition comprising at least one surfactant chosen from anionic and/or nonionic surfactants, at least one silicone compound, at least one non-silicone fatty compound, optionally at least one polymer, optionally at least one hydrogen peroxide stabilizing compound, optionally at least one antioxidant, and at least one solvent. In other embodiments, a first compartment or container comprises a hair care or treatment composition such as a shampoo or conditioner, and a second compartment or container comprises a composition that can be used in methods according to the disclosure.

### DETAILED DESCRIPTION

The disclosure relates to compositions and methods for treating hair. The compositions are acidic and comprise hydrogen peroxide and one or more additional components such as surfactants, silicones, fatty compounds other than silicones, polymers, pigments and/or direct dyes, amino acids and/or amino sulfonic acids, vitamins, pH adjusters, solvents, etc. The methods comprise applying the compositions according to the disclosure to the hair to treat the hair.

### I. Compositions

The hair treatment compositions comprise hydrogen peroxide. In various embodiments, it may be useful to choose a hydrogen peroxide generating agent, which can generate hydrogen peroxide in situ. For example, useful hydrogen peroxide generating agents may be chosen from urea peroxide; polymeric complexes that can release hydrogen peroxide such as polyvinylpyrrolidone/H₂O₂ and other polymeric complexes described in U.S. Pat. Nos. 5,008,093; 3,376,110; and 5,183,901; and/or oxidases which produce hydrogen peroxide, for example glucose oxidase or uricase. In all instances herein, the term “hydrogen peroxide” is intended to include hydrogen peroxide generating agents unless expressly stated otherwise. In some embodiments, however, only hydrogen peroxide is used and the compositions do not contain any other hydrogen peroxide generating agents.

The hair treatment compositions may comprise up to about 10% hydrogen peroxide, such as from about 1% to about 8%, from about 1.5% to about 6%, from about to about 4%, or from about 2.5% to about 3.5% by weight, based on the total weight of the composition. For example, the hair treatment compositions may comprise hydrogen peroxide in an amount ranging from about 0.1% to about 10%, from about 0.1% to about 9%, from about 0.1% to about 8%, from about 0.1% to about 7%, from about 0.1% to about 6%, from about 0.1% to about 5%, from about 0.1% to about 4%, from about 0.1% to about 3%, from about 0.1% to about 2%, from about 0.1% to about 1%, from about 0.5% to about 10%, from about 0.5% to about 9%, from about 0.5% to about 8%, from about 0.5% to about 7%, from about 0.5% to about 6%, from about 0.5% to about 5%, from about 0.5% to about 4%, from about 0.5% to about 3%, from about 0.5% to about 2%, from about 0.5% to about 1%, from about 1% to about 10%, from about 1% to about 9%, from about 1% to about 8%, from about 1% to about 7%, from about 1% to about 6%, from about 1% to about 5%, from about 1% to about 4%, from about 1% to about 3%, from about 1% to about 2%, from about 1.5% to about 10%, from about 1.5% to about 9%, from about 1.5% to about 8%, from about 1.5% to about 7%, from about 1.5% to about 6%, from about 1.5% to about 5%, from about 1.5% to about 4%, from about 1.5% to about 3%, from about 1.5% to about 2%, from about 2% to about 10%, from about 2% to about 9%, from about 2% to about 8%, from about 2% to about 7%, from about 2% to about 6%, from about 2% to about 5%, from about 2% to about 4%, from about 2% to about 3%, from about 2.5% to about 10%, from about 2.5% to about 9%, from about 2.5% to about 8%, from about 2.5% to about 7%, from about 2.5% to about 6%, from about 2.5% to about 5%, from about 2.5% to about 4%, from about 2.5% to about 3%, from about 3% to about 10%, from about 3% to about 9%, from about 3% to about 8%, from about 3% to about 7%, from about 3% to about 6%, from about 3% to about 5%, from about 3% to about 4%, from about 3.5% to about 10%, from about 3.5% to about 9%, from about 3.5% to about 8%, from about 3.5% to about 7%, from about 3.5% to about 6%, from about 3.5% to about 5%, from about 3.5% to about 4%, from about 4% to about 10%, from about 4% to about 9%, from about 4% to about 8%, from about 4% to about 7%, from about 4% to about 6%, from about 4% to about 5%, from about 4.5% to about 10%, from about 4.5% to about 9%, from about 4.5% to about 8%, from about 4.5% to about 7%, from about 4.5% to about 6%, from about 4.5% to about 5%, from about 5% to about 10%, from about 5% to about 9%, from about 5% to about 8%, from about 5% to about 7%, from about 5% to about 6%, from about 5.5% to about 10%, from about 5.5% to about 9%, from about 5.5% to about 8%, from about 5.5% to about 7%, from about 5.5% to about 6%, from about 6% to about 10%, from about 6% to about 9%, from about 6% to about 8%, or from about 6% to about 7% by weight, relative to the total weight of the composition. In various embodiments, the compositions may include about 0.5%, about 0.75%, about 1%, about 1.25%, about 1.5%, about 1.75%, about 2%, about 2.25%, about 2.5%, about 2.75%, about 3%, about 3.25%, about 3.5%, about 3.75%, about 4%, about 4.25%, about 4.5%, about 4.75%, about 5%, about 5.25%, about 5.5%, about 5.75%, about 6%, about 6.25%, about 6.5%, about 6.75%, about 7%, about 7.25%, about 7.5%, about 7.75%, or about 8% hydrogen peroxide by weight, relative to the total weight of the composition, including all ranges and subranges using any of the foregoing as upper and lower limits.

In some embodiments, it may be particularly advantageous that the compositions comprise no less than about 1.5% hydrogen peroxide, such as at least 1.50% exactly, at least about 1.6%, at least about 1.7%, at least about 1.8%, at least about 1.9%, or at least about 2% by weight, relative to the total weight of the composition. In other embodiments, it may be particularly useful that the compositions comprise no more than about 8%, such as no more than about 7%, no more than about 6%, or no more than about 5% hydrogen peroxide by weight, relative to the total weight of the composition.

Although not required, it may in some embodiments be helpful to include at least one hydrogen peroxide stabilizer, for example at least one phosphate compound such as tetrasodium etidronate or tetrasodium pyrophosphate, and/or sodium salicylate. If present, the hydrogen peroxide stabilizers are typically present in amounts less than about 1%, for example can be included in amounts ranging from about 0.01% to about 0.75% by weight, relative to the total weight of the composition.

In addition to hydrogen peroxide, the hair treatment compositions can include additional components such as surfactants, silicones, fatty compounds other than silicones, and/or polymers, which have been found to impart stability to the compositions and/or provide cosmetic benefits to the treated hair.

### Surfactants

Hair treatment compositions according to the disclosure can comprise one or more surfactants, although in at least some embodiments the compositions may be free or essentially free of surfactants. Useful surfactants include cationic, anionic, nonionic, and amphoteric surfactants. Without intending to be limited by theory, the surfactants, in particular anionic and/or non-ionic surfactants, surprisingly impart stability to the compositions in the presence of hydrogen peroxide. In some preferred embodiments, the hair treatment compositions comprise at least one surfactant chosen from anionic and/or non-ionic surfactants. In other preferred embodiments, the hair treatment compositions comprise at least one anionic surfactant and at least one nonionic surfactant.

In various embodiments, the total amount of surfactants can range up to about 15%, such as from about 0.01% to about 15%, from about 0.1% to about 12%, from about 0.5% to about 10%, or from about 1% to about 8% by weight, relative to the total weight of the composition. For example, the total amount of surfactants may range from about 0.1% to about 15%, from about 0.1% to about 12%, from about 0.1% to about 10%, from about 0.1% to about 9%, from about 0.1% to about 8%, from about 0.1% to about 7%, from about 0.1% to about 6%, from about 0.1% to about 5%, from about 0.1% to about 4%, from about 0.1% to about 3%, from about 0.5% to about 15%, from about 0.5% to about 12%, from about 0.5% to about 10%, from about 0.5% to about 9%, from about 0.5% to about 8%, from about 0.5% to about 7%, from about 0.5% to about 6%, from about 0.5% to about 5%, from about 0.5% to about 4%, from about 0.5% to about 3%, from about 1% to about 15%, from about 1% to about 12%, from about 1% to about 10%, from about 1% to about 9%, from about 1% to about 8%, from about 1% to about 7%, from about 1% to about 6%, from about 1% to about 5%, from about 1% to about 4%, from about 1% to about 3%, from about 1.5% to about 15%, from about 1.5% to about 12%, from about 1.5% to about 10%, from about 1.5% to about 9%, from about 1.5% to about 8%, from about 1.5% to about 7%, from about 1.5% to about 6%, from about 1.5% to about 5%, from about 1.5% to about 4%, from about 1.5% to about 3%, from about 2% to about 15%, from about 2% to about 12%, from about 2% to about 10%, from about 2% to about 9%, from about 2% to about 8%, from about 2% to about 7%, from about 2% to about 6%, from about 2% to about 5%, from about 2% to about 4%, from about 2% to about 3%, from about 2.5% to about 15%, from about 2.5% to about 12%, from about 2.5% to about 10%, from about 2.5% to about 9%, from about 2.5% to about 8%, from about 2.5% to about 7%, from about 2.5% to about 6%, from about 2.5% to about 5%, from about 2.5% to about 4%, from about 2.5% to about 3%, from about 3% to about 15%, from about 3% to about 12%, from about 3% to about 10%, from about 3% to about 9%, from about 3% to about 8%, from about 3% to about 7%, from about 3% to about 6%, from about 3% to about 5%, from about 3% to about 4%, from about 3.5% to about 15%, from about 3.5% to about 12%, from about 3.5% to about 10%, from about 3.5% to about 9%, from about 3.5% to about 8%, from about 3.5% to about 7%, from about 3.5% to about 6%, from about 3.5% to about 5%, from about 3.5% to about 4%, from about 4% to about 15%, from about 4% to about 12%, from about 4% to about 10%, from about 4% to about 9%, from about 4% to about 8%, from about 4% to about 7%, from about 4% to about 6%, from about 4% to about 5%, from about 4.5% to about 15%, from about 4.5% to about 12%, from about 4.5% to about 10%, from about 4.5% to about 9%, from about 4.5% to about 8%, from about 4.5% to about 7%, from about 4.5% to about 6%, from about 4.5% to about 5%, from about 5% to about 15%, from about 5% to about 12%, from about 5% to about 10%, from about 5% to about 9%, from about 5% to about 8%, from about 5% to about 7%, or from about 5% to about 6% by weight, relative to the total weight of the composition.

### Nonionic Surfactants

By way of example only, nonionic surfactants that can be chosen include alcohols, alpha-diols, alkylphenols, and esters of fatty acids, these compounds being ethoxylated, propoxylated, or glycerolated and having at least one fatty chain comprising, for example, from 8 to 18 carbon atoms, it being possible for the number of ethylene oxide or propylene oxide groups to range from 2 to 50, and for the number of glycerol groups to range from 1 to 30. Non-limiting mention may also be made of copolymers of ethylene oxide and/or of propylene oxide; condensates of ethylene oxide and/or of propylene oxide with fatty alcohols; polyethoxylated fatty amides comprising, for example, from 2 to 30 mol of ethylene oxide; polyglycerolated fatty amides comprising, for example, from 1.5 to 5 glycerol groups, such as from 1.5 to 4; ethoxylated fatty acid esters of sorbitan comprising from 2 to 30 mol of ethylene oxide; ethoxylated oils from plant origin; fatty acid esters of sucrose; fatty acid esters of polyethylene glycol; polyethoxylated fatty acid mono or diesters of glycerol (C6-C24)alkylpolyglycosides; N—(C6-C24)alkylglucamine derivatives, amine oxides such as (C10-C14)alkylamine oxides or N—(C10-C14)acylaminopropylmorpholine oxides; lauryl alcohol containing 4 mol of glycerol (INCI name: Polyglyceryl-4 Lauryl Ether), lauryl alcohol containing 1.5 mol of glycerol, oleyl alcohol containing 4 mol of glycerol (INCI name: Polyglyceryl-4 Oleyl Ether), oleyl alcohol containing 2 mol of glycerol (INCI name: Polyglyceryl-2 Oleyl Ether), cetearyl alcohol containing 2 mol of glycerol, cetearyl alcohol containing 6 mol of glycerol, oleocetyl alcohol containing 6 mol of glycerol, and octadecanol containing 6 mol of glycerol; and combinations of two or more thereof. Sorbitol esters of C8-C24 fatty acids and alkoxylated derivatives thereof can also be chosen, for example sorbitan palmitate, sorbitan trioleate, and esters of fatty acids and alkoxylated sorbitan containing for example from 20 to 100 EO, such as for example polysorbate 80 or polysorbate 85.

In some embodiments, ethoxylated fatty alcohols may be chosen, such as deceth-3, trideceth-3, trideceth-5, trideceth-6, trideceth-10, trideceth-12, laureth-2 (a lauryl ethoxylate having an average degree of ethoxylation of 2), laureth-3 (a lauryl ethoxylate having an average degree of ethoxylation of 3), laureth-23 (a lauryl ethoxylate having an average degree of ethoxylation of 23), ceteth-10 (a cetyl alcohol ethoxylate having an average degree of ethoxylation of 10) steareth-10 (a stearyl alcohol ethoxylate having an average degree of ethoxylation of 10), steareth-2 (a stearyl alcohol ethoxylate having an average degree of ethoxylation of 2), steareth-6 (a stearyl alcohol ethoxylate having an average degree of ethoxylation of 6), steareth-100 (a stearyl alcohol ethoxylate having an average degree of ethoxylation of 100), beheneth-5 (a behenyl alcohol ethoxylate having an average degree of ethoxylation of 5), beheneth-10 (a behenyl alcohol ethoxylate having an average degree of ethoxylation of 10), and mixtures thereof.

Other examples of nonionic surfactants that may be used include fatty acid alkanolamides (isopropanolamides of fatty acids) such as cocoamide MIPA, lauramide MIPA, linoleamide MIPA, myristamide MIPA, oleamide MIPA, stearamide MIPA, isostearamide MIPA, and mixtures thereof.

Other useful nonionic surfactants include Poloxamers, which are polymers made of a block of polyoxyethylene, followed by a block of polyoxypropylene, followed by a block of polyoxyethylene, such as Poloxamer 101, Poloxamer 105, Poloxamer 108, Poloxamer 122, Poloxamer 123, Poloxamer 124, Poloxamer 181, Poloxamer 182, Poloxamer 183, Poloxamer 184, Poloxamer 185, Poloxamer 188, Poloxamer 212, Poloxamer 215, Poloxamer 217, Poloxamer 231, Poloxamer 234, Poloxamer 235, Poloxamer 237, Poloxamer 238, Poloxamer 282, Poloxamer 284, Poloxamer 288, Poloxamer 331, Poloxamer 333, Poloxamer 334, Poloxamer 335, Poloxamer 338, Poloxamer 401, Poloxamer 402, Poloxamer 403, Poloxamer 407, Poloxamer 105 Benzoate, Poloxamer 182 Dibenzoate. By way of example, Poloxamer 184, a hydrophobic chain of polyoxypropylene with two hydrophilic chains of polyoxyethylene, may be chosen.

If present, the total amount of nonionic surfactants may range up to about 10%, such as up to about 9%, up to about 8%, up to about 7%, up to about 6%, up to about 5%, up to about 4%, up to about 3.5%, up to about 3%, up to about 2.5%, up to about 2%, up to about 1.5%, up to about 1%, or up to about 0.5% by weight, relative to the total weight of the composition. For example, the total amount of nonionic surfactants may range from about 0.001% to about 8%, from about 0.01% to about 7%, from about 0.1% to about 6%, or from about 0.5% to about 5% by weight, relative to the total weight of the composition. In some embodiments, the compositions are free or essentially free of nonionic surfactants.

### Cationic Surfactants

Non-limiting examples of useful cationic surfactants include polyoxyalkylenated primary, secondary, or tertiary fatty amine salts, quaternary ammonium salts, and mixtures thereof. In some embodiments, compositions may include one or more tetraalkylammonium halides or alkyltrimethylammonium halides, for example those whose alkyl group includes from 12 to 22 carbon atoms, such as from 14 to 20 carbon atoms. In some embodiments, compositions may include chloride or methyl sulfate salts of diacyloxyethyldimethylammonium, diacyloxyethylhydroxyethylmethylammonium, monoacyloxyethyldihydroxyethylmethylammonium, triacyloxyethylmethylammonium, and/or monoacyloxyethylhydroxyethyldimethylammonium.

By way of example, behentrimonium chloride, cetrimonium chloride, behenalkonium chloride, benzethonium chloride, cetylpyridinium chloride, behentrimonium chloride, lauralkonium chloride, cetalkonium chloride, cetrimonium bromide, cetrimonium chloride, cethylamine hydrofluoride, chlorallylmethenamine chloride (Quaternium-15), distearyldimonium chloride (Quaternium-5), dodecyl dimethyl ethylbenzyl ammonium chloride (Quaternium-14), Quaternium-22, Quaternium-26, Quaternium-18 hectorite, dimethylaminoethylchloride hydrochloride, cysteine hydrochloride, diethanolammonium POE (10) oletyl ether phosphate, diethanolammonium POE (3)oleyl ether phosphate, tallow alkonium chloride, dimethyl dioctadecylammoniumbentonite, stearalkonium chloride, domiphen bromide, denatonium benzoate, myristalkonium chloride, laurtrimonium chloride, ethylenediamine dihydrochloride, guanidine hydrochloride, pyridoxine HCl, iofetamine hydrochloride, meglumine hydrochloride, methylbenzethonium chloride, myrtrimonium bromide, oleyltrimonium chloride, polyquaternium-1, procainehydrochloride, cocobetaine, stearalkonium bentonite, stearalkoniumhectonite, stearyl trihydroxyethyl propylenediamine dihydrofluoride, tallowtrimonium chloride, hexadecyltrimethyl ammonium bromide, stearamidopropyl dimethylamine, or combinations of two or more thereof may be chosen.

If present, the total amount of cationic surfactants may range up to about 5%, such as up to about 4%, up to about 3.5%, up to about 3%, up to about 2.5%, up to about 2%, up to about 1.5%, up to about 1%, or up to about 0.5% by weight, relative to the total weight of the composition. For example, the total amount of cationic surfactants may range from about 0.001% to about 5%, from about 0.005% to about 3%, or from about 0.01% to about 2.5% by weight, relative to the total weight of the composition. In at least some embodiments, the compositions are free or substantially free of cationic surfactants. In some embodiments, the compositions are free or essentially free of behentrimonium chloride and/or cetrimonium chloride. In some embodiments the compositions are free or essentially free of cationic surfactants.

### Anionic Surfactants

Non-limiting examples of anionic surfactants that can be used include alkyl sulfates, alkyl ether sulfates, acyl isethionates, acyl glycinates, acyl taurates, acyl amino acids, acyl sarcosinates, sulfosuccinates, sulfonates, and mixtures of two or more thereof, for example those where the alkyl and acyl groups comprise from 6 to 24 carbon atoms. For example, anionic surfactant may be chosen from alkylamido ether sulfates, alkylaryl polyether sulfates, monoglyceride sulfates, alkylsulfonates, alkylamide sulfonates, alkylarylsulfonates, alpha-olefin sulfonates, paraffin sulfonates, alkylsulfosuccinates, alkyl ether sulfosuccinates, alkylamide sulfosuccinates, alkyl sulfoacetates, acylsarcosinates, acylglutamates, alkylsulfosuccinamates, acylisethionates and N-acyltaurates, salts of alkyl monoesters and polyglycoside-polycarboxylic acids, acyllactylates, salts of D-galactoside uronic acids, salts of alkyl ether carboxylic acids, salts of alkyl aryl ether carboxylic acids, and salts of alkylamido ether carboxylic acids; or the non-salified forms of all of these compounds, for example where the alkyl and acyl groups contain from 6 to 24 carbon atoms and the aryl group denoting a phenyl group. When the anionic surfactant(s) are in salt form, they may be chosen, for example, from alkali metal salts such as the sodium or potassium salt, ammonium salts, amine salts such as amino alcohol salts, or alkaline-earth metal salts such as magnesium salts. The aforementioned compounds may optionally be oxyethylenated, for example with 1 to 50 ethylene oxide units. For example, the anionic surfactant may comprise, consist essentially of, or consist of sulfonates such as sodium olefin sulfonates, e.g. sodium C14-C16 olefin sulfonate.

If present, the total amount of anionic surfactants may range up to about 10%, such as up to about 9%, up to about 8%, up to about 7%, up to about 6%, up to about 5%, up to about 4%, up to about 3.5%, up to about 3%, up to about 2.5%, up to about 2%, up to about 1.5%, up to about 1%, or up to about 0.5% by weight, relative to the total weight of the composition. For example, the total amount of anionic surfactants may range from about 0.01% to about 6%, from about 0.05% to about 4%, from about 0.1% to about 3%, or from about 0.5% to about 2.5% by weight, relative to the total weight of the composition. It may, in some embodiments, be advantageous to choose an amount of anionic surfactant that would not be considered an amount sufficient to impart cleansing effects to the hair, i.e. would be lower than a cleansing amount. In some embodiments, the hair treatment compositions are free or essentially free of anionic surfactants.

In some preferred embodiments, the anionic surfactant is not a sulfate surfactant. Thus, in at least some preferred embodiments, the hair treatment compositions are free or essentially free of sulfates. For example, the hair treatment compositions may contain less than about 5%, such as less than about 4.5%, less than about 4%, less than about 3.5%, less than about 3%, less than about 2.5%, less than about 2%, less than about 1.5%, less than about 1%, less than about 0.5%, or less than about 0.1% of sulfates by weight, relative to the total weight of the hair treatment composition.

### Amphoteric Surfactants

In some embodiments, useful amphoteric surfactants include derivatives of aliphatic secondary and tertiary amines where the aliphatic radical can be a straight or branched chain and one of the aliphatic substituents contains from about 8 to about 18 carbon atoms and one contains an anionic group such as carboxy, sulfonate, sulfate, phosphate, or phosphonate. Exemplary amphoteric surfactants include sodium cocaminopropionate, sodium cocaminodipropionate, sodium cocoamphoacetate, sodium cocoamphohydroxypropylsulfonate, sodium cocoamphopropionate, sodium cornamphopropionate, sodium lauraminopropionate, sodium lauroamphoacetate, sodium lauroamphohydroxypropylsulfonate, sodium lauroamphopropionate, sodium cornamphopropionate, sodium lauriminodipropionate, ammonium cocaminopropionate, ammonium cocaminodipropionate, ammonium cocoamphoacetate, ammonium cocoamphohydroxypropylsulfonate, ammonium cocoamphopropionate, ammonium cornamphopropionate, ammonium lauraminopropionate, ammonium lauroamphoacetate, ammonium lauroamphohydroxypropylsulfonate, ammonium lauroamphopropionate, ammonium cornamphopropionate, ammonium lauriminodipropionate, triethanonlamine cocaminopropionate, triethanonlamine cocaminodipropionate, triethanonlamine cocoamphoacetate, triethanonlamine cocoamphohydroxypropylsulfonate, triethanonlamine cocoamphopropionate, triethanonlamine cornamphopropionate, triethanonlamine lauraminopropionate, triethanonlamine lauroamphoacetate, triethanonlamine lauroamphohydroxypropylsulfonate, triethanonlamine lauroamphopropionate, triethanonlamine cornamphopropionate, triethanonlamine lauriminodipropionate, cocoamphodipropionic acid, disodium caproamphodiacetate, disodium caproamphoadipropionate, disodium capryloamphodiacetate, disodium capryloamphodipriopionate, disodium cocoamphocarboxyethylhydroxypropylsulfonate, disodium cocoamphodiacetate, disodium cocoamphodipropionate, disodium dicarboxyethylcocopropylenediamine, disodium laureth-5 carboxyamphodiacetate, disodium lauriminodipropionate, disodium lauroamphodiacetate, disodium lauroamphodipropionate, disodium oleoamphodipropionate, disodium PPG-2-isodecethyl-7 carboxyamphodiacetate, lauraminopropionic acid, lauroamphodipropionic acid, lauryl aminopropylglycine, and lauryl diethylenediaminoglycine, as well as mixtures of two or more thereof.

Betaines may also be used. For example, coco dimethyl carboxymethyl betaine, lauryl dimethyl carboxymethyl betaine, lauryl dimethyl alphacarboxyethyl betaine, cetyl dimethyl carboxymethyl betaine, cetyl dimethyl betaine, lauryl bis-(2-hydroxyethyl) carboxymethyl betaine, stearyl bis-(2-hydroxypropyl) carboxymethyl betaine, oleyl dimethyl gamma-carboxypropyl betaine, lauryl bis-(2-hydroxypropyl) alphacarboxyethyl betaine, coco dimethyl sulfopropyl betaine, stearyl dimethyl sulfopropyl betaine, lauryl dimethyl sulfoethyl betaine, lauryl bis-(2-hydroxyethyl) sulfopropyl betaine, oleyl betaine, or cocamidopropyl betaine (cocobetaine) may be chosen, as well as mixtures of two or more thereof.

If present, the total amount of amphoteric surfactants may range up to about 10%, such as up to about 9%, up to about 8%, up to about 7%, up to about 6%, up to about 5%, up to about 4%, up to about 3.5%, up to about 3%, up to about 2.5%, up to about 2%, up to about 1.5%, up to about 1%, or up to about 0.5% by weight, relative to the total weight of the composition. For example, the total amount of amphoteric surfactants may range from about 0.001% to about 6%, from about 0.01% to about 4%, from about 0.1% to about 3%, or from about 0.5% to about 2% by weight, relative to the total weight of the composition. In at least some embodiments, the compositions are free or substantially free of amphoteric surfactants.

In preferred embodiments, it may be particularly advantageous to choose combinations of surfactants that provide stability to the composition. For example, the hair treatment compositions may comprise at least one nonionic surfactant and at least one anionic surfactant. In further embodiments, the hair treatment compositions comprise at least one nonionic surfactant, at least one anionic surfactant, and at least one amphoteric surfactant. In still further embodiments, the hair treatment compositions comprise at least one nonionic surfactant, at least one anionic surfactant, at least one amphoteric surfactant, and at least one cationic surfactant. In some embodiments, the hair treatment compositions comprise laureth-2 as the sole surfactant, or comprise laureth-2 in addition to one or more other surfactants such as sulfonates, e.g. sodium C14-C16 olefin sulfonate. For example, the compositions may comprise from about 1% to about 6%, such as from about 1.5% to about 6%, from about 2% to about 6%, or from about 2% to about 5% of laureth-2 and/or sodium C14-C16 olefin sulfonate by weight, relative to the total weight of the composition. In at least some embodiments, the hair treatment compositions are free or essentially free of behentrimonium chloride.

In preferred embodiments, the compositions comprise one or more surfactants chosen from laureth-2, lauryl alcohol, deceth-3, trideceth-3, trideceth-5, trideceth-6, trideceth-10, trideceth-12, steareth-6, oleyl alcohol, cocamide MIPA, cocobetaine, behentrimonium chloride, cetrimonium chloride, polysorbate 80, sodium C14-C16 sulfonate, and/or Poloxamer 184.

### Silicones

Compositions according to the disclosure may optionally include at least one silicone compound, although in at least some embodiments the compositions may be free or essentially free of silicones. Silicones that can be used include, for example, amine-functionalized silicones (e.g., amodimethicone), dimethicone, derivatives of dimethicone, or combinations thereof. Without intending to be limited by theory, the silicone compound(s) surprisingly imparts stability to the compositions, particularly over time and with regard to texture and appearance.

In some embodiments, the silicone compound may be chosen from dimethicone, amodimethicone, cyclomethicone, polysilicone-11, phenyl trimethicone, trimethylsilylamodimethicone, stearoxytrimethylsilane, derivatives of dimethicone (e.g. alkoxylated derivatives), or combinations thereof. For example, the composition may comprise at least one silicone chosen from dimethicone, amodimethicone, PEG-7 dimethicone, PEG-8 dimethicone, PEG-9 dimethicone, PEG-10 dimethicone, PEG-12 dimethicone, PEG-14 dimethicone, PEG-17 dimethicone, PEG/PPG-3/10 dimethicone, PEG/PPG-4/12 dimethicone, PEG/PPG-17/18 dimethicone, cetyl PEG/PPG-10/1 dimethicone, dimethicone PEG-8 benzoate, dimethicone PEG-7 phosphate, dimethicone PEG-8 phosphate, dimethicone PEG-10 phosphate, or a combination of two or more thereof.

As further examples, the silicone may be chosen from polydimethylsiloxanes comprising alkyl or alkoxy groups which are pendent and/or at the end of the silicone chain, which groups each contain from 2 to 24 carbon atoms, or phenyl silicones, such as phenyl trimethicones, phenyl dimethicones, phenyl(trimethylsiloxy)diphenylsiloxanes, diphenyl dimethicones, diphenyl (methyldiphenyl)trisiloxanes, or (2-phenylethyl) trimethylsiloxysilicates.

Examples of useful derivatives of dimethicone include those comprising at least one oxyethylene group and at least one oxypropylene group, such as bis-PEG/PPG-14/14 dimethicone, bis-isobutyl PEG/PPG-10/7 dimethicone copolymer, bis-PEG/PPG-18/6 dimethicone; bis-PEG/PPG-20/20 dimethicone, bis-PEG/PPG-16/16 dimethicone, cetyl PEG/PPG-15/16 butyl ether dimethicone, cetyl PEG/PPG-15/15 butyl ether dimethicone, cetyl PEG/PPG-7/3 dimethicone, cetyl PEG/PPG-10/1 dimethicone, dimethicone PEG/PPG-7/4 phosphate, dimethicone PEG/PPG-12/4 phosphate, PEG/PPG-28/21 acetate dimethicone, PEG/PPG-22/22 butyl ether dimethicone, PEG/PPG-23/23 butyl ether dimethicone, PEG/PPG-24/18 butyl ether dimethicone, PEG/PPG-3/10 dimethicone, PEG/PPG-4/12 dimethicone, PEG/PPG-6/11 dimethicone, PEG/PPG-8/14 dimethicone, PEG/PPG-12/16 dimethicone, PEG/PPG-12/18 dimethicone, PEG/PPG-14/4 dimethicone, PEG/PPG-15/5 dimethicone, PEG/PPG-15/15 dimethicone, PEG/PPG-16/2 dimethicone, PEG/PPG-16/8 dimethicone, PEG/PPG-17/18 dimethicone, PEG/PPG-18/12 dimethicone, PEG/PPG-19/19 dimethicone, PEG/PPG-20/6 dimethicone, PEG/PPG-20/15 dimethicone, PEG/PPG-20/20 dimethicone, PEG/PPG-20/29 dimethicone, PEG/PPG-22/23 dimethicone, PEG/PPG-22/24 dimethicone, PEG/PPG-25/25 dimethicone, PEG/PPG-27/27 dimethicone, PEG/PPG-30/10 dimethicone, PEG/PPG-10/3 oleyl ether dimethicone, or mixtures of two or more thereof.

In some embodiments, the hair treatment compositions comprise dimethicone, amodimethicone, PEG/PPG-4/12 dimethicone, or a combination of two or more thereof as the silicone compound(s).

It may in at least some embodiments be advantageous to choose a combination of at least two silicone compounds, where a first and second silicone compounds have different molecular weights.

The hair treatment compositions may comprise a total amount of silicones up to about 10%, such as from about 1% to about 8%, from about 1.5% to about 6%, from about 2% to about 4%, or from about 2.5% to about 3.5% by weight, based on the total weight of the composition. For example, the hair treatment compositions may comprise a total amount of silicones ranging from about 0.1% to about 10%, from about 0.1% to about 9%, from about 0.1% to about 8%, from about 0.1% to about 7%, from about 0.1% to about 6%, from about 0.1% to about 5%, from about 0.1% to about 4%, from about 0.1% to about 3%, from about 0.1% to about 2%, from about 0.1% to about 1%, from about 0.5% to about 10%, from about 0.5% to about 9%, from about 0.5% to about 8%, from about 0.5% to about 7%, from about 0.5% to about 6%, from about 0.5% to about 5%, from about 0.5% to about 4%, from about 0.5% to about 3%, from about 0.5% to about 2%, from about 0.5% to about 1%, from about 1% to about 10%, from about 1% to about 9%, from about 1% to about 8%, from about 1% to about 7%, from about 1% to about 6%, from about 1% to about 5%, from about 1% to about 4%, from about 1% to about 3%, from about 1% to about 2%, from about 1.5% to about 10%, from about 1.5% to about 9%, from about 1.5% to about 8%, from about 1.5% to about 7%, from about 1.5% to about 6%, from about 1.5% to about 5%, from about 1.5% to about 4%, from about 1.5% to about 3%, from about 1.5% to about 2%, from about 2% to about 10%, from about 2% to about 9%, from about 2% to about 8%, from about 2% to about 7%, from about 2% to about 6%, from about 2% to about 5%, from about 2% to about 4%, from about 2% to about 3%, from about 2.5% to about 10%, from about 2.5% to about 9%, from about 2.5% to about 8%, from about 2.5% to about 7%, from about 2.5% to about 6%, from about 2.5% to about 5%, from about 2.5% to about 4%, from about 2.5% to about 3%, from about 3% to about 10%, from about 3% to about 9%, from about 3% to about 8%, from about 3% to about 7%, from about 3% to about 6%, from about 3% to about 5%, from about 3% to about 4%, from about 3.5% to about 10%, from about 3.5% to about 9%, from about 3.5% to about 8%, from about 3.5% to about 7%, from about 3.5% to about 6%, from about 3.5% to about 5%, from about 3.5% to about 4%, from about 4% to about 10%, from about 4% to about 9%, from about 4% to about 8%, from about 4% to about 7%, from about 4% to about 6%, from about 4% to about 5%, from about 4.5% to about 10%, from about 4.5% to about 9%, from about 4.5% to about 8%, from about 4.5% to about 7%, from about 4.5% to about 6%, from about 4.5% to about 5%, from about 5% to about 10%, from about 5% to about 9%, from about 5% to about 8%, from about 5% to about 7%, from about 5% to about 6%, from about 5.5% to about 10%, from about 5.5% to about 9%, from about 5.5% to about 8%, from about 5.5% to about 7%, from about 5.5% to about 6%, from about 6% to about 10%, from about 6% to about 9%, from about 6% to about 8%, or from about 6% to about 7% by weight, relative to the total weight of the composition. In various embodiments, the compositions may include about 0.5%, about 0.75%, about 1%, about 1.25%, about 1.5%, about 1.75%, about 2%, about 2.25%, about 2.5%, about 2.75%, about 3%, about 3.25%, about 3.5%, about 3.75%, about 4%, about 4.25%, about 4.5%, about 4.75%, about 5%, about 5.25%, about 5.5%, about 5.75%, about 6%, about 6.25%, about 6.5%, about 6.75%, about 7%, about 7.25%, about 7.5%, about 7.75%, or about 8% of silicones by weight, relative to the total weight of the composition, including all ranges and subranges using any of the foregoing as upper and lower limits.

### Non-Silicone Fatty Compounds

Hair treatment compositions optionally comprise one or more fatty compounds other than silicones, although in at least some embodiments the compositions may be free or essentially free of non-silicone fatty compounds. As used herein, “fatty compound” means an organic compound insoluble in water at normal temperature (25° C.) and at atmospheric pressure (760 mmHg) (solubility below 5% and such as below 1% and further such as below 0.1%). Without intending to be limited by theory, the non-silicone fatty compounds surprisingly impart stability to the compositions, particularly over time and with regard to texture and appearance.

Fatty compounds that may be chosen include, for example, linear or branched hydrocarbons, fatty alcohols, esters of fatty acids, esters of fatty alcohols, oils such as mineral, plant, animal and synthetic non-silicone oils, and waxes such as waxes from plants, although it is contemplated that one or more of the foregoing may be excluded from the hair treatment compositions.

Non-limiting examples of linear or branched hydrocarbons that can be chosen include polybutene, hydrogenated polyisobutene, polyisoprene, polydecenes such as hydrogenated polydecene, and/or linear, branched, and/or cyclic alkanes which are optionally volatile, such as, for example, isohexadecane, isododecane, isodecane, or isohexadecane.

The fatty alcohols that can be chosen include, but are not limited to, alkoxylated or non-alkoxylated, saturated or unsaturated, linear or branched, fatty alcohols. The fatty alcohol may be chosen from, for example, C6-C30 alcohols, C8-C30 alcohols, C12-C30 alcohols, C9-C11 alcohols, C12-C13 alcohols, C12-C15 alcohols, C12-C16 alcohols, C14-C15 alcohols, or C12-C22 alcohols, such as arachidyl alcohol, behenyl alcohol, caprylic alcohol, cetearyl alcohol, cetyl alcohol, coconut alcohol, decyl alcohol, hydrogenated tallow alcohol, jojoba alcohol, lauryl alcohol, myristyl alcohol, oleyl alcohol, palm alcohol, palm kernel alcohol, stearyl alcohol, tallow alcohol, tridecyl alcohol, or combinations of two or more thereof.

Non-limiting examples of fatty acids that may be used include optionally branched and/or unsaturated fatty acids such as myristic acid, palmitic acid, stearic acid, behenic acid, oleic acid, linoleic acid, linolenic acid, isostearic acid, or mixtures of two or more thereof.

As used herein, plant oils are oils derived from a plant, for example, oils from seeds or fruits. Non-limiting examples of plant oils include coconut oil, soybean oil, canola oil, rapeseed oil, corn oil, cottonseed oil, olive oil, palm oil, peanut oil, safflower oil, sesame oil, sunflower oil, linseed oil, palm kernel oil, tung oil, jatropha oil, mustard oil, camelina oil, pennycress oil, castor oil, wheatgerm oil, apricot kernel oil, pistachio oil, poppy oil, pine oil, avocado oil, hazel nut oil, grapeseed oil, colza oil, cade oil, peach kernel oil, coffee bean oil, jojoba oil, and a mixture of two or more thereof. In some embodiments, the hair treatment composition includes apricot kernel oil and/or soybean oil.

Non-limiting examples of waxes that can be used include beeswax, hydrogenated alkyl olive esters, carnauba wax, candelilla wax, ouricoury wax, Japan wax, cork fibre wax or sugar cane wax, rice wax, rice bran wax. montan wax, paraffin wax, lignite wax or microcrystalline wax, ceresin or ozokerite, palm kernel glycerides/hydrogenated palm glycerides, palm butter, sumac wax, citrus aurantium dulcis (orange) peel wax, theobroma grandiflorum seed butter, Helianthus annuus (sunflower) seed wax, siliconyl candellila wax, Chinese wax, cetyl palmitate, lanolin, shellac, spermaceti, cetyl esters, hydrogenated castor wax; triglyceride esters such as tribehenin (glyceryl tribehenate); synthetic waxes such as those of the hydrocarbon type and polyethylene waxes obtained from the polymerization or copolymerization of ethylene, polypropylene waxes, or mixtures of two or more thereof. In some embodiments, the hair treatment composition includes candelilla wax.

In some preferred embodiments, the hair treatment compositions comprise one or more plant oils. The hair treatment compositions in other preferred embodiments comprise one or more non-silicone fatty compounds chosen from oleyl alcohol, lauryl alcohol, candelilla wax, apricot oil, and/or soybean oil.

The total amount of non-silicone fatty compounds may vary but is typically up to about 10%, such as from about 0.01% to about 10%, from about 0.1% to about 8%, from about 0.25% to about 5%, or from about 0.5% to about 5% by weight, relative to the total weight of the composition. For example, the total amount of non-silicone fatty compounds may range from about 0.1% to about 10%, from about 0.1% to about 9%, from about 0.1% to about 8%, from about 0.1% to about 7%, from about 0.1% to about 6%, from about 0.1% to about 5%, from about 0.1% to about 4%, from about 0.1% to about 3%, from about 0.1% to about 2%, from about 0.1% to about 1%, from about 0.5% to about 10%, from about 0.5% to about 9%, from about 0.5% to about 8%, from about 0.5% to about 7%, from about 0.5% to about 6%, from about 0.5% to about 5%, from about 0.5% to about 4%, from about 0.5% to about 3%, from about 0.5% to about 2%, from about 0.5% to about 1%, from about 1% to about 10%, from about 1% to about 9%, from about 1% to about 8%, from about 1% to about 7%, from about 1% to about 6%, from about 1% to about 5%, from about 1% to about 4%, from about 1% to about 3%, from about 1% to about 2%, from about 1.5% to about 10%, from about 1.5% to about 9%, from about 1.5% to about 8%, from about 1.5% to about 7%, from about 1.5% to about 6%, from about 1.5% to about 5%, from about 1.5% to about 4%, from about 1.5% to about 3%, from about 1.5% to about 2%, from about 2% to about 10%, from about 2% to about 9%, from about 2% to about 8%, from about 2% to about 7%, from about 2% to about 6%, from about 2% to about 5%, from about 2% to about 4%, from about 2% to about 3%, from about 2.5% to about 10%, from about 2.5% to about 9%, from about 2.5% to about 8%, from about 2.5% to about 7%, from about 2.5% to about 6%, from about 2.5% to about 5%, from about 2.5% to about 4%, from about 2.5% to about 3%, from about 3% to about 10%, from about 3% to about 9%, from about 3% to about 8%, from about 3% to about 7%, from about 3% to about 6%, from about 3% to about 5%, from about 3% to about 4%, from about 3.5% to about 10%, from about 3.5% to about 9%, from about 3.5% to about 8%, from about 3.5% to about 7%, from about 3.5% to about 6%, from about 3.5% to about 5%, from about 3.5% to about 4%, from about 4% to about 10%, from about 4% to about 9%, from about 4% to about 8%, from about 4% to about 7%, from about 4% to about 6%, from about 4% to about 5%, from about 4.5% to about 10%, from about 4.5% to about 9%, from about 4.5% to about 8%, from about 4.5% to about 7%, from about 4.5% to about 6%, or from about 4.5% to about 5% by weight, relative to the total weight of the composition.

### Polymers

In some embodiments, the hair treatment composition optionally further comprises a polymer, which may, for example, be a thickening polymer and/or conditioning polymer, although in at least some embodiments the compositions may be free or essentially free of such polymers.

Polymers that may be chosen include, in various embodiments, quaternary ammonium polymers and polysaccharide polymers. For example, useful polymers include cellulose and derivatives of cellulose, such as, for example hydroxyethylcellulose, hydroxymethylcellulose, methylhydroxyethylcellulose, hydroxypropylcellulose, hydroxybutylcellulose, hydroxyethylmethylcellulose, hydroxypropylmethylcellulose, and cetyl hydroxyethylcellulose. Guars may also be chosen, for example hydroxypropyl guar, guar hydroxypropyltrimonium chloride, hydroxypropyl guar hydroxypropyltrimonium chloride, and guar hydroxypropyltrimethylammonium chloride. Starches such as hydroxypropyl starch phosphate or potato starch (modified or unmodified) may also be chosen. Polyquaterniums can also be included, for example polyquaternium-1, polyquaternium-2, polyquaternium-3, polyquaternium-4, polyquaternium-5, polyquaternium-6, polyquaternium-7, polyquaternium-8, polyquaternium-9, polyquaternium-10, polyquaternium-11, polyquaternium-12, polyquaternium-13, polyquaternium-14, polyquaternium-15, polyquaternium-16, polyquaternium-17, polyquaternium-18, polyquaternium-19, polyquaternium-20, polyquaternium-21, polyquaternium-22, polyquaternium-23, polyquaternium-24, polyquaternium-25, polyquaternium-26, polyquaternium-27, polyquaternium-28, polyquaternium-29, polyquaternium-30, polyquaternium-40, polyquaternium-41, polyquaternium-42, polyquaternium-43, polyquaternium-44, polyquaternium-45, polyquaternium-46, polyquaternium-47, polyquaternium-48, polyquaternium-49, polyquaternium-50, polyquaternium-51, polyquaternium-52, polyquaternium-53, polyquaternium-54, polyquaternium-55, polyquaternium-56, polyquaternium-57, polyquaternium-58, polyquaternium-59, polyquaternium-60, polyquaternium-61, polyquaternium-62, polyquaternium-63, polyquaternium-64, polyquaternium-65, polyquaternium-66, and polyquaternium-67. Additionally, carbomers, which are hydrophilic polymers of acrylic acid cross-linked with polyalkenyl ethers or divinyl glycol or homopolymers of acrylic acid cross-linked with allyl ethers of sucrose or pentaerytritol, for example those available as the Carbopol 900 series (e.g., Carbopol 954), may also be used. In some embodiments, the polymer is chosen from polyvinylpyrrolidone and copolymers thereof, such as polyvinylpyrrolidone (PVP)/vinyl acetate (PVP/VA copolymer), polyvinylpyrrolidone (PVP)/eicosene copolymer, and PVP/hexadecene copolymer.

The total amount of polymers, e.g. thickening and/or conditioning polymers, may vary, but is typically up to about 15%, such as from about 0.01% to about 10%, from about 0.05% to about 8%, from about 0.1% to about 10%, or from about 0.25% to about 8% by weight, relative to the total weight of the composition. For example, the total amount of non-silicone fatty compounds may range from about 0.05% to about 10%, from about 0.05% to about 9%, from about 0.05% to about 8%, from about 0.05% to about 7%, from about 0.05% to about 6%, from about 0.05% to about 5%, from about 0.05% to about 4%, from about 0.05% to about 3%, from about 0.05% to about 2%, from about 0.05% to about 1%, from about 0.1% to about 10%, from about 0.1% to about 9%, from about 0.1% to about 8%, from about 0.1% to about 7%, from about 0.1% to about 6%, from about 0.1% to about 5%, from about 0.1% to about 4%, from about 0.1% to about 3%, from about 0.1% to about 2%, from about 0.1% to about 1%, from about 0.5% to about 10%, from about 0.5% to about 9%, from about 0.5% to about 8%, from about 0.5% to about 7%, from about 0.5% to about 6%, from about 0.5% to about 5%, from about 0.5% to about 4%, from about 0.5% to about 3%, from about 0.5% to about 2%, from about 0.5% to about 1%, from about 1% to about 10%, from about 1% to about 9%, from about 1% to about 8%, from about 1% to about 7%, from about 1% to about 6%, from about 1% to about 5%, from about 1% to about 4%, from about 1% to about 3%, from about 1% to about 2%, from about 1.5% to about 10%, from about 1.5% to about 9%, from about 1.5% to about 8%, from about 1.5% to about 7%, from about 1.5% to about 6%, from about 1.5% to about 5%, from about 1.5% to about 4%, from about 1.5% to about 3%, from about 1.5% to about 2%, from about 2% to about 10%, from about 2% to about 9%, from about 2% to about 8%, from about 2% to about 7%, from about 2% to about 6%, from about 2% to about 5%, from about 2% to about 4%, from about 2% to about 3%, from about 2.5% to about 10%, from about 2.5% to about 9%, from about 2.5% to about 8%, from about 2.5% to about 7%, from about 2.5% to about 6%, from about 2.5% to about 5%, from about 2.5% to about 4%, from about 2.5% to about 3%, from about 3% to about 10%, from about 3% to about 9%, from about 3% to about 8%, from about 3% to about 7%, from about 3% to about 6%, from about 3% to about 5%, from about 3% to about 4%, from about 3.5% to about 10%, from about 3.5% to about 9%, from about 3.5% to about 8%, from about 3.5% to about 7%, from about 3.5% to about 6%, from about 3.5% to about 5%, from about 3.5% to about 4%, from about 4% to about 10%, from about 4% to about 9%, from about 4% to about 8%, from about 4% to about 7%, from about 4% to about 6%, from about 4% to about 5%, from about 4.5% to about 10%, from about 4.5% to about 9%, from about 4.5% to about 8%, from about 4.5% to about 7%, from about 4.5% to about 6%, or from about 4.5% to about 5% by weight, relative to the total weight of the composition.

In preferred embodiments, the hair treatment compositions comprise at least one carbomer, and the total amount of carbomers is less than about 10%, such as less than about 7.5%, or less than about 5%, for example ranges from about 0.01% to about 5%, such as from about 0.1% to about 5%, from about 0.1% to about 4%, from about 0.1% to about 3%, from about 0.1% to about 2%, from about 0.1% to about 1%, from about 0.25% to about 5%, from about 0.25% to about 4%, from about 0.25% to about 3% by weight, from about 0.25% to about 2%, or from about 0.25% to about 1% by weight, relative to the total weight of the composition.

### Pigments/Direct Dyes

It may be desirable to impart non-permanent color to the hair with hair treatment compositions according to the disclosure. Therefore, in some embodiments, the hair treatment compositions may optionally comprise one or more hair coloring agents chosen from pigments and/or direct dyes. However, in at least some embodiments, the hair treatment compositions do not impart color to the hair, and therefore do not contain pigments and/or direct dyes in a quantity sufficient to alter the color of the hair. In some embodiments, the hair treatment compositions are free or substantially free of pigments and/or free or substantially free of direct dyes.

In various embodiments, hair treatment compositions comprise one or more direct dyes chosen from anionic direct dyes, cationic direct dyes, non-ionic direct dyes, or mixtures of two or more thereof. For example, the direct dyes may be chosen from nitrophenylenediamines, nitroaminophenols, azo dyes, anthraquinones, triarylmethane dyes, or indophenols, as well as salts thereof. Direct dyes that are acid-base indicators may be chosen.

As suitable anionic (acid) direct dyes, for example, mention may be made of Acid Yellow 1 (D&C Yellow 7, Citronin A, Ext. D&C Yellow No. 7, Japan Yellow 403, CI 10316, COLIPA no B001), Acid Yellow 3 (COLIPA no: C 54, D&C Yellow No 10, Quinoline Yellow, E104, Food Yellow 13), Acid Yellow 9 (CI 13015), Acid Yellow 17 (CI 18965), Acid Yellow 23 (COLIPA no C. 29, Covacap Jaune W 1100 (LCW), Sicovit Tartrazine 85 E 102 (BASF), Tartrazine, Food Yellow 4, Japan Yellow 4, FD&C Yellow No. 5), Acid Yellow 36 (CI 13065), Acid Yellow 121 (CI 18690), Acid Orange 6 (CI 14270), Acid Orange 7 (2-Naphthol orange, Orange II, CI 15510, D&C Orange 4, COLIPA no C015), Acid Orange 10 (CI 16230; Orange G sodium salt), Acid Orange 11 (CI 45370), Acid Orange 15 (CI 50120), Acid Orange 20 (CI 14600), Acid Orange 24 (BROWN 1; CI 20170; KATSU201; no sodium salt; Brown No. 201; RESORCIN BROWN; ACID ORANGE 24; Japan Brown 201; D & C Brown No. 1), Acid Red 14 (C114720), Acid Red 18 (E124, Red 18; CI 16255), Acid Red 27 (E 123, CI 16185, C-Rot 46, Echtrot D, FD&C Red Nr. 2, Food Red 9, Naphtholrot S), Acid Red 33 (Red 33, Fuchsia Red, D&C Red 33, CI 17200), Acid Red 35 (CI C118065), Acid Red 51 (CI 45430, Pyrosin B, Tetraiodofluorescein, Eosin J, lodeosin), Acid Red 52 (CI 45100, Food Red 106, Solar Rhodamine B, Acid Rhodamine B, Red no 106 Pontacyl Brilliant Pink), Acid Red 73 (CI CI 27290), Acid Red 87 (Eosin, CI 45380), Acid Red 92 (D&C Red Red 104; AKA231; RED 28; SUREDYE; 11969 Red; PHLOXINE; CI 45405; CI 45410; EOSINE B); Acid Red 95 (CI 45425, Erythrosine, Simacid Erythrosine Y), Acid Red 184 (CI 15685), Acid Red 195; Pigment Red 57:1 (E180; D & CRED7 CI 15850; Rubine 4BN; CI 15850:1; PIGMENT RED 57; Litholrubine BK; LITHOLRUBINE RB; LITHOLRUBINE BCA; Lithol Rubine B); Acid Violet 43 (Jarocol Violet 43, Ext. D&C Violet no 2, CI 60730, COLIPA no C063), Acid Violet 49 (CI 42640), Acid Violet 50 (CI 50325), Acid Blue 1 (Patent Blue, CI 42045), Acid Blue 3 (Patent Blue V, CI 42051), Acid Blue 7 (CI 42080), Acid Blue 104 (CI 42735), Acid Blue 9 (E 133, Patent Blue AE, Amido Blue AE, Erioglaucine A, CI 42090, CI Food Blue 2), Acid Blue 62 (CI 62045), Acid Blue 74 (E 132, CI 73015), Acid Blue 80 (CI 61585), Acid Green 3 (CI 42085, Foodgreenl), Acid Green 5 (CI 42095), Acid Green 9 (CI 42100), Acid Green 22 (CI 42170), Acid Green 25 (CI 61570, Japan Green 201, D&C Green No. 5), Acid Green 50 (Brilliant Acid Green BS, CI 44090, Acid Brilliant Green BS, E 142), Acid Black 1 (Black no 401, Naphthalene Black 10B, Amido Black 10B, CI 20470, COLIPA no B15), Acid Black 52 (CI 15711), Food Yellow 8 (CI 14270), Food Blue 5, D&C Yellow 8, D&C Green 5, D&C Orange 10, D&C Orange 11, D&C Red 21, D&C Red 27, D&C Red 33, D&C Brown 1, bromophenol blue, tetrabromophenol blue, and mixtures of two or more thereof.

Non-limiting examples of suitable cationic (basic) dyes include, for example, Basic Blue 6 (CI—No. 51,175), Basic Blue 7 (CI—No. 42,595) Basic Blue 9 (CI—No. 52,015), Basic Blue 26 (CI—No. 44,045), Basic Blue 41 (CI—No. 11,154), Basic Blue 99 (CI—No. 56,059), HC Blue 15, HC Blue 16 (Blue Quat Bromide), Cationic Blue 347, Basic Brown 4 (CI—No. 21,010), Basic Brown 16 (CI—No. 12,250), Basic Brown 17 (CI—No. 12,251), Natural Brown 7 (CI—No. 75,500), Basic Green 1 (CI—No. 42,040), Basic Red 2 (CI—No. 50,240), Basic Red 22 (CI—No. 11,055), Basic Red 51, Basic Red 76 (CI—No. 12,245), Basic Violet 1 (CI—No. 42,535), Basic Violet 2, Basic Violet 3 (CI—No. 42,555), Basic Violet 10 (CI—No. 45,170), Basic Violet 14 (CI—No. 42,510), Basic Yellow 57 (CI—No. 12,719), Basic Yellow 87, Basic Orange 31, and mixtures of two or more thereof.

Non-limiting examples of non-ionic direct dyes that can be used include HC Yellow 2, HC Yellow 4, HC Yellow 5, HC Yellow 6, HC Yellow 12, HC Orange 1, Disperse Orange 3, HC Red 1, HC Red 3, HC Red 7, HC Red 10, HC Red 11, HC Red 13, HC Red BN, HC Blue 2, HC Blue 11, HC Blue 12, HC Blue 15, Disperse Blue 3, HC Violet 1, Disperse Violet 1, Disperse Violet 4, Disperse Black 9, 1,4-diamino-2-nitrobenzene, 2-amino-4-nitrophenol, 1,4-bis(2-hydroxyethyl)-amino-2-nitrobenzene, 3-nitro-4-(2-hydroxyethyl) aminophenol, 2-(2-hydroxyethyl) amino-4,6-dinitrophenol, 4-[(2-hydroxyethyl)amino]-3-nitro-1-methylbenzene, 1-amino-4-(2-hydroxyethyl)amino-5-chloro-2-nitrobenzene, 4-amino-3-nitrophenol, 1-(2′-ureidoethyl)amino-4-nitrobenzene, 2-[(4-amino-2-nitrophenyl)amino]benzoic acid, 4-[(3-hydroxypropyl)amino]-3-nitrophenol, 4-nitro-o-phenylenediamine, 6-nitro-1,2,3,4-tetrahydroquinoxaline, 2-hydroxy-1,4-naphthoquinone, picramic acid and its salts, N,N′-bis(2-hydroxyethyl)-2-nitro-p-phenylenediamine, 2-amino-6-chloro-4-nitropheneol, 4-ethylamino-3-nitrobenzoic acid, 2-chloro-6-ethylamino-4-nitrophenol, and mixtures of two or more thereof.

In further embodiments, hair treatment compositions according to the disclosure may comprise one or more pigments. The pigments may be coated or uncoated.

By way of non-limiting example, azo pigments containing one or more azo groups may be chosen from those of formula A-N═N—B, where A is chosen from an optionally substituted (hetero)aryl, B is chosen from optionally substituted (hetero)aryl or —CH[C(O)—R]—C(O)—X1-A′, and A′ represents an optionally substituted (hetero)aryl and R represents a hydrogen atom or a group (C₁-C₆)alkyl, wherein the groups A, A′, and B may optionally be (hetero)aryls that do not contain any solubilizing groups such as —SO3H or —COOH. Exemplary useful pigments may, for example, include monoazo pigments such as β-naphthols, monoazopyrrolones, benzimidazolone pigments; diazo pigments such as diazodiarylide pigments and bis(N-acetoacetarylide); triazo; and tetraazo pigments. Mention may also be made of azo metal complex pigments.

In yet further exemplary embodiments, other pigments such as isoindolinone and isoindoline pigments, phthalocyanin pigments; quinacridone pigments; perinone pigments; perylene pigments; anthraquinone pigments such as hydroxyanthraquinone pigments; aminoanthraquinone pigments including acylaminoanthraquinones and azo anthraquinone pigments; heterocyclic anthraquinones; polycarboxylic anthraquinone pigments, pyranthrone pigments; anthranthrone pigments; diketopyrrolopyrrole (DPP) pigments; thioindigo pigments; dioxazine pigments; triphenylmethane pigments; quinophthalone pigments; or fluorescent pigments may be chosen.

In still further embodiments, carbon black, titanium oxide, chromium oxide, pigments of D&C or FD&C type and lakes thereof, and especially those known under the names D&C Blue No. 4, D&C Brown No. 1, FD&C Green No. 3, D&C Green No. 5, D&C Green No. 6, FD&C Green No. 8, D&C Orange No. 4, D&C Orange No. 5, D&C Orange No. 10, D&C Orange No. 11, FD&C Red No. 4, D&C Red No. 6, D&C Red No. 7, D&C Red No. 17, D&C Red No. 21, D&C Red No. 22, D&C Red No. 27, D&C Red No. 28, D&C Red No. 30, D&C Red No. 31, D&C Red No. 33, D&C Red No. 34, D&C Red No. 36, FD&C Red No. 40, FD&C Red 40 lake, D&C Violet No. 2, FD & C Blue No. 1, D&C Yellow No. 6, FD&C Yellow No. 6, D&C Yellow No. 7, Ext. D&C Yellow No. 7, D&C Yellow No. 8, D&C Yellow No. 10, or D&C Yellow No. 11 may be chosen.

In various embodiments, the hair coloring agent(s) present are limited to direct dyes. In further embodiments, the hair coloring agent(s) present are limited to pigments. In still further embodiments, the hair coloring agent(s) present include combinations of direct dyes and pigments. In preferred embodiments, the hair coloring compositions do not include oxidative dyes, i.e. the hair treatment compositions are free or essentially free of oxidation dye compounds.

If present, the total amount of pigments and/or direct dyes in the hair treatment compositions ranges from about 0.001% to about 10% by weight, such as from about 0.01% to about 10%, about 0.1% to about 10%, about 0.001% to about 5%, about 0.01% to about 5%, about 0.1% to about 5%, about 0.001% to about 4%, about 0.01% to about 4%, about 0.1% to about 4%, about 0.001% to about 3%, about 0.01% to about 3%, about 0.1% to about 3%, about 0.001% to about 2.5%, about 0.01% to about 2.5%, about 0.1% to about 2.5%, about 0.001% to about 2%, about 0.01% to about 2%, about 0.1% to about 2%, about 0.001% to about 1.5%, about 0.01% to about 1.5%, about 0.1% to about 1.5%, about 0.001% to about 1%, about 0.01% to about 1%, or about 0.1% to about 1% by weight, of the total weight of the hair treatment composition.

### Solvents

The hair treatment compositions according to the disclosure comprise at least one solvent. The solvent may be chosen from water, non-aqueous solvents, or combinations thereof. The total amount of solvent is generally less than about 98%, for example may range from about 10% to about 95% by weight, relative to the total weight of the hair treatment composition. In preferred embodiments, the total amount of solvent may range from about 20% to about 90%, about 20% to about 85%, about 20% to 75%, about 20% to 70%, about 20% to about 65%, about 20% to about 60%, about 20% to about 55%, about 30% to about 90%, about 30% to about 85%, about 30% to 75%, about 30% to 70%, about 30% to about 65%, about 30% to about 60%, about 30% to about 55%, about 40% to about 90%, about 40% to about 85%, about 40% to 75%, about 40% to 70%, about 40% to about 65%, about 40% to about 60%, about 40% to about 55%, about 50% to about 90%, about 50% to about 85%, about 50% to 75%, about 50% to 70%, about 50% to about 65%, or about 50% to about 60% by weight, relative to the total weight of the hair treatment composition.

In some embodiments, the solvent comprises, consists essentially of, or consists of water. In some embodiments, the total amount of water ranges from about 30% to about 95%, from about 50% to about 90%, or from about 60% to about 85% by weight, relative to the total weight of the hair treatment composition.

In preferred embodiments, the solvent comprises at least one non-aqueous solvent. For example, non-aqueous solvents that can be used include C1-4 alcohols, fatty alcohols, fatty ethers, fatty esters, polyols, glycols, vegetable oils, mineral oils, liposomes, laminar lipid materials, or combinations thereof. Non-limiting examples of useful non-aqueous solvents include alkanediols or alkanetriols such as glycerin, 1,2,6-hexanetriol, trimethylolpropane, ethylene glycol, propylene glycol, diethylene glycol, triethylene glycol, tetraethylene glycol, pentaethylene glycol, dipropylene glycol, 2-butene-1,4-diol, 2-ethyl-1,3-hexanediol, 2-methyl-2,4-pentanediol, caprylyl glycol, 1,2-hexanediol, 1,2-pentanediol, and 4-methyl-1,2-pentanediol; alkyl alcohols having 1 to 4 carbon atoms such as ethanol, methanol, butanol, propanol, and isopropanol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, and dipropylene glycol mono-iso-propyl ether; 2-pyrrolidone, N-methyl-2-pyrrolidone, 1,3-dimethyl-2-imidazolidinone, formamide, acetamide, dimethyl sulfoxide, sorbit, sorbitan, acetine, diacetine, triacetine, sulfolane, or combinations thereof.

If present, the total amount of the non-aqueous solvents in the composition may range from about 0.1% to about 20%, such as from about 0.5% to about 18%, from about 1% to about 15%, or from about 5% to about 15% by weight, relative to the total weight of the composition.

In some preferred embodiments, the solvent comprises water and at least one non-aqueous solvent. In some embodiments, the composition comprises water and at least one non-aqueous solvent chosen from propylene glycol, dipropylene glycol, tripropylene glycol, glycerin, and/or PPG-2 butyl ether.

### PH Adjusters

The hair treatment composition is generally acidic, and may range from about 2 to about 7, such as from about 2.5 to about 6.5, from about 3 to about 5, from about 3 to about 4.5, or from about 3.5 to about 4.5. In some embodiments, the composition has a pH value strictly less than 7, such as strictly less than 6 or strictly less than 5, for example ranging from about 2 to no greater than 6, from about 2 to no greater than 5.5, from about 2 to no greater than 5, from about 2.5 to no greater than 6, from about 2.5 to no greater than 5.5, from about 2.5 to no greater than 5, from about 3 to no greater than 6, from about 3 to no greater than 5.5, or from about 3 to no greater than 5. In various embodiments, the hair treatment compositions have a pH of about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0, about 4.1, about 4.2, about 4.3, about 4.4, about 4.5, about 4.6, about 4.7, about 4.8, about 4.9, or about 5.0, or may be in any range of from about 2.5 to about 5.0, using any of the foregoing as lower and upper limits.

Therefore, the composition may optionally comprise one or more pH adjusters such as hydrochloric acid, (ortho)phosphoric acid, sulfuric acid, boric acid, or carboxylic acids such as acetic acid, lactic acid, or citric acid, or sulfonic acid, or salts of any of the foregoing. The pH adjuster(s) may be present in an amount needed to adjust the pH of the composition to the desired pH, such as up to about 1%, up to about 0.5% up to about 0.1%, or from about 0.001% to about 1%, about 0.01% to about 1%, or about 0.01% to about 0.5% by weight of the first composition.

### Amino Acids and Amino Sulfonic Acids

The hair treatment composition may optionally comprise at least one amino acid and/or at least one amino sulfonic acid, although in at least some embodiments the compositions are free or essentially free of amino acids and/or amino sulfonic acids. Salts of amino acids and amino sulfonic acids may also be chosen, and are expressly included whether or not so stated.

Amino acids that may be chosen include, for example, alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine. Exemplary amino sulfonic acids include aminomethane sulfonic acid, 2-aminoethane sulfonic acid (taurine), aminopropane sulfonic acid, aminobutane sulfonic acid, aminohexane sulfonic acid, aminoisopropyl sulfonic acid, aminododecyl sulfonic acid, aminobenzene sulfonic acid, aminotoulene sulfonic acid, sulfanilic acid, chlorosulfanilic acid, diamino benzene sulfonic acid, amino phenol sulfonic acid, amino propyl benzene sulfonic acid, and amino hexyl benzene sulfonic acid. In some embodiments, the hair treatment compositions comprise at least one amino acid and at least one amino sulfonic acid. In preferred embodiments, the composition comprises arginine, serine, and/or taurine. For example, in some embodiments, arginine, serine, and/or taurine are the only amino acids and amino sulfonic acids present in the hair treatment compositions.

If present, the total amount of amino acid(s) and/or amino sulfonic acid(s) may range from about 0.001% to about 5%, such as from about 0.005% to about 3%, or from about 0.01% to about 2% by weight, relative to the total weight of the hair color composition. For example, in some embodiments, the total amount of the at least one amino acid and/or amino sulfonic acid may range from about 0.05% to about 5%, such as from about 0.05% to about 4%, from about 0.05% to about 3%, from about 0.05% to about 2.5%, from about 0.05% to about 2%, from about 0.05% to about 1.5%, from about 0.05% to about 1%, from about 0.05% to about 0.5%, from about 0.1% to about 5%, from about 0.1% to about 4%, from about 0.1% to about 3%, from about 0.1% to about 2.5%, from about 0.1% to about 2%, from about 0.1% to about 1.5%, from about 0.1% to about 1%, or from about 0.1% to about 0.5% by weight, relative to the total weight of the hair color composition.

### Additional Components

The composition according to the disclosure may optionally comprise any auxiliary or additional component suitable for use in the hair treatment compositions. Such components may include, but are not limited to, dyes/pigments for coloring the composition, film forming agents, humectants, fillers, structuring agents, propellants, hydrogen peroxide stabilizers, antioxidants or reducing agents, penetrants, sequestrants, fragrances, buffers, dispersants, ceramides, vitamins (e.g. tocopherol), preserving agents, opacifiers, sunscreen agents, and antistatic agents, although it should be understood that the composition may be free or essentially free of any of the foregoing.

In various embodiments, the additional components may each be present in the hair treatment compositions in an amount ranging from about 0.001% to about 10%, such as about 0.01% to about 5%, about 0.01% to about 4%, about 0.01% to about 3%, about 0.01% to about 2%, about 0.01% to about 1%, about 0.01% to about 0.75%, about 0.01% to about 0.5%, about 0.01% to about 0.4%, about 0.01% to about 0.3%, about 0.01% to about 0.2%, or about 0.01% to about 0.1% by weight, relative to the total weight of the hair treatment composition. For example, in some embodiments the compositions may comprise at least one hydrogen peroxide stabilizer, wherein the total amount of hydrogen peroxide stabilizers ranges from about 0.001% to about 10%, preferably from about 0.01% to about 5%, more preferably from about 0.01% to about 3%, more preferably still from about 0.01% to about 1.5%, most preferably from about 0.01% to about 1% by weight, relative to the total weight of the composition. As a further example, the compositions may comprise at least one antioxidant, wherein the total amount of antioxidants ranges from about 0.001% to about 10%, preferably from about 0.01% to about 5%, more preferably from about 0.01% to about 3%, more preferably still from about 0.01% to about 1.5%, most preferably from about 0.01% to about 1% by weight, relative to the total weight of the composition.

The hair treatment compositions according to the disclosure may be in any form, but are advantageously in the form of a cream, a gel, a gel cream, an emulsion, or a lotion. In various embodiments, the compositions may have a viscosity ranging from about 1000 mPa-s to about 5000 mPa-s, such as from about 2000 mPa-s to about 4000 mPa-s, from about 2500 mPa-s to about 3750 mPa-s, from about 2750 mPa-s to about 3500 mPa-s, or from about 3000 mPa-s to about 3250 mPa-s, when measured at 25° C., 200 RPM for 30 seconds using a spindle M3 (Rheomat 180).

In preferred embodiments, the hair treatment compositions are compositions for imparting shine (and optionally non-permanent color) to hair. For example, the hair treatment compositions may be compositions for imparting shine (and optionally non-permanent color) to hair that has previously been dyed or bleached. In addition, the hair treatment compositions are in “ready-to-use” or “all-in-one” form, in that they are not required to be mixed with a separate composition prior to use. The ready-to-use compositions are surprisingly stable over time even though they contain hydrogen peroxide, in that they exhibit no or minimal change in viscosity and/or degradation of hydrogen peroxide for at least one day (24 hours), at least two days (48 hours), at least three days (72 hours), at least one week, at least two weeks, at least four weeks, at least eight weeks, or even at least six months when stored at 4° C., 25° C., 37° C., or 45° C. For example, the hair treatment composition may exhibit less than 10%, such as less than 8%, less than 5%, or even less than 3% change in hydrogen peroxide content and/or viscosity (measured at 25° C., 200 RPM for 30 seconds using a spindle M3 (Rheomat 180)) when stored at a particular temperature for a particular period of time.

### II. Methods

The disclosure further relates to methods of using the compositions described herein to treat hair by applying the compositions to the hair. Hair treated with the compositions surprisingly demonstrates enhanced shine, conditioning, smoothness, strength, ease of detangling or combability, less frizz, and/or appears and feels healthier than hair not treated with the compositions.

The hair treatment compositions may be applied to hair that is wet, damp, or dry. In some embodiments it is preferred to apply the composition to dry or substantially dry hair, and in other embodiments it is preferred to apply the composition to wet or damp hair. By way of example, it may be particularly beneficial to apply the hair treatment compositions to hair that has recently been chemically reshaped (e.g. by straightening or by permanent waving), lightened, highlighted, and/or dyed. For example, a salon service where hair is chemically reshaped, lightened, highlighted, and/or dyed may include a subsequent step of applying a hair treatment composition to the hair during the same visit. For example, the hair may be treated with a hair treatment composition according to the disclosure within about 6 hours or less, such as about 4 hours or less, about 2 hours or less, about 1 hour or less, or about 30 minutes or less, after a chemical reshaping, lightening, and/or dyeing service.

In some embodiments, the hair may be treated with a composition that imparts alkalinity to the hair immediately or substantially immediately prior to being treated with a composition according to the disclosure. Thus, in some embodiments, the hair to which a hair treatment composition is applied is alkaline. Such a treatment may be advantageous to both lower the pH of, and impart shine (and optionally non-permanent color) to, the hair. By way of non-limiting example, the hair may have undergone a chemical treatment such as a bleaching, oxidative dyeing, permanent waving, relaxing, and/or straightening process, and optionally rinsed, immediately or substantially immediately prior to a composition according to the disclosure being applied to the hair. In such embodiments, the prior chemical treatment process would be understood to leave the hair in an alkaline state, even after the chemical processing composition was rinsed from the hair. However, because hair treatment compositions according to the disclosure are generally acidic, treating the alkaline hair with such hair treatment compositions has the benefit of lowering the pH of the hair, while simultaneously imparting shine (and optionally non-permanent color) to the hair.

Without intending to be limited by theory, it is believed that such a method of treating hair may also be beneficial when certain direct dyes are included in the hair treatment composition, e.g. those that impart different colors at different pH values (e.g. dyes that are considered to be acid-base indicators), in order to achieve a desired hair color. As a non-limiting example, tetrabromophenol blue is known to impart a yellow color below a pH of about 4.5-4.6, and a blue color above that value. Therefore, if a blue hair color is desired, a hair treatment composition according to the disclosure comprising tetrabromophenol blue can be applied to hair that is alkaline.

As another example, treatment with a composition according to the disclosure may be a separate, stand-alone treatment that a consumer may seek in order to “refresh” the appearance of the hair between chemical reshaping, lightening, and/or dyeing services. For example, the hair may be treated with a hair treatment composition according to the disclosure more than about 24 hours, such as more than about 48 hours more than about 72 hours, more than about one week, more than about two weeks, or more than about one month after such chemical reshaping, lightening, and/or oxidative dyeing service. Such treatments may provide the hair with a shiny, healthy appearance and/or feel despite having undergone one or more harsh chemical processes.

In some embodiments, the hair treatment composition may be applied to hair that is wet, for example hair that has just been washed and/or rinsed. However, it should be understood that hair treatment compositions according to the disclosure are not cleansing compositions, i.e. the compositions are not shampoos, and the methods are not methods of cleansing the hair.

Compositions according to the disclosure are generally considered rinse-out compositions, however it should be understood that any manner of removing the composition from the treated hair is contemplated. Thus, the composition can be removed from the treated hair after a desired leave-in time, for example the hair may be rinsed with water.

One particularly surprising aspect of the disclosure is that the leave-in or “processing” time needed to impart the advantageous benefits described herein to treated hair is generally less than needed for other conditioning, shine-imparting, etc., compositions. For example, although the processing time required for similar compositions is generally as much as 15 or 20 minutes, compositions according to the disclosure impart the same or even better properties to the hair in about 10 minutes or less, such as about 5 minutes or less. Therefore, methods according to the disclosure may include a leave-in time of less than 20 minutes, for example from at least about 15 seconds such as at least about 30 seconds or at least about 1 minute, to less than about 15 minutes, such as less than about 12 minutes, less than about 10 minutes, less than about 8 minutes, less than about 6 minutes, for example no more than about 5 minutes. In preferred embodiments, the leave-in time is less than about 15 minutes, for example may range from about 1 minute to about 15 minutes, from about 2 minutes to about 12 minutes, from about 5 minutes to about 10 minutes, from about 1 minute to about 10 minutes, from about 1 minute to about 8 minutes, from about 1 minute to about 5 minutes, from about 2 minutes to about 10 minutes, from about 2 minutes to about 8 minutes, from about 2 minutes to about 5 minutes, from about 3 minutes to about 10 minutes, from about 3 minutes to about 8 minutes, or from about 3 minutes to about 5 minutes.

In various embodiments, the disclosure relates to methods of treating hair, methods of imparting shine to hair, methods of improving the appearance and/or feel of hair, methods of improving ease of detangling and/or combing of hair, methods of reducing the appearance of split ends, and/or methods of non-permanently altering the color of hair comprising applying an effective amount of a composition according to the disclosure to the hair and leaving the composition on the hair for a processing time ranging from at least about 15 seconds such as at least about 30 seconds or at least about 1 minute to about 15 minutes, from at least about 15 seconds such as at least about 30 seconds or at least about 1 minute to about 12 minutes, from at least about 15 seconds such as at least about 30 seconds or at least about 1 minute to about 10 minutes, from at least about 15 seconds such as at least about 30 seconds or at least about 1 minute to about 8 minutes, from at least about 15 seconds such as at least about 30 seconds or at least about 1 minute to about 5 minutes, from about 2 minutes to about 15 minutes, from about 2 minutes to about 12 minutes, from about 2 minutes to about 10 minutes, from about 2 minutes to about 8 minutes, or from about 2 minutes to about 5 minutes, or for about 30 seconds, for about 1 minute, for about 2 minutes, for about 3 minutes, for about 4 minutes, for about 5 minutes, for about 6 minutes, for about 7 minutes, for about 8 minutes, for about 9 minutes, or for about 10 minutes, and subsequently removing the composition from the hair, e.g. by rinsing the hair.

Such reduced processing time is believed to be particularly advantageous in that it may reduce or minimize damage to the hair caused by the treatment process, as well as minimizing any unintended lightening of the hair color caused by the treatment process. Thus, in some embodiments methods according to the disclosure are those described which do not visibly lighten or lift the color of the hair.

### III. Kits

The disclosure further relates to kits. Kits according to the disclosure comprise at least one, such as at least two, compartments or containers, and optionally further comprise instructions.

In various embodiments, a first compartment or container comprises hydrogen peroxide or a hydrogen peroxide generating agent, preferably in a solvent such as water, and a second compartment or container comprises a composition as described herein other than the hydrogen peroxide or hydrogen peroxide generating agent. For example, a second compartment or container may comprise a composition comprising at least one surfactant chosen from anionic and/or nonionic surfactants, at least one silicone compound, at least one non-silicone fatty compound, optionally at least one polymer, optionally at least one hydrogen peroxide stabilizing compound, optionally at least one antioxidant, optionally at least one amino and/or amino sulfonic acid, and at least one solvent.

In further embodiments, a first compartment or container comprises a hair care or treatment composition such as a shampoo or conditioner composition, and a second compartment or container comprises a composition as described herein.

Kits may come in any form and comprise any type of compartments or containers. For example, a useful kit may be a dual-chamber device that is configured to dispense amounts of compositions from each chamber upon activation. Other useful kits may comprise ampoules. It should be understood that the type of kit is not limited.

Having described the many embodiments of the present invention in detail, it will be apparent that modifications and variations are possible without departing from the scope of the disclosure defined in the appended claims. Furthermore, it should be appreciated that all examples in the present disclosure, while illustrating many embodiments of the disclosure, are provided as non-limiting examples and are, therefore, not to be taken as limiting the various aspects so illustrated. It is to be understood that all definitions herein are provided for the present disclosure only.

In this application, the use of the singular includes the plural unless specifically stated otherwise. The singular forms “a,” “an,” “the,” and “at least one” are understood to encompass the plural as well as the singular unless the context clearly dictates otherwise. The expression “one or more” means “at least one” and thus includes individual components as well as mixtures/combinations. Likewise, the term “a salt thereof” also relates to “salts thereof.” Thus, where the disclosure refers to “an element selected from the group consisting of A, B, C, D, E, F, a salt thereof, or mixtures thereof,” it indicates that that one or more of A, B, C, D, and F may be included, one or more of a salt of A, a salt of B, a salt of C, a salt of D, a salt of E, or a salt of F may be included, or a mixture of any two of A, B, C, D, E, F, a salt of A, a salt of B, a salt of C, a salt of D, a salt of E, and a salt of F may be included.

The term “and/or” should be understood to include both the conjunctive and the disjunctive. For example, “amino acids and/or salts thereof” means “amino acids and salts thereof” as well as “amino acids or salts thereof,” and expressly covers instances of either.

As used herein, the term “salts” refers to throughout the disclosure may include salts having a counterion such as an alkali metal, alkaline earth metal, or ammonium counterion. This list of counterions, however, is non-limiting. Salts also include a dissociated form of a compound, e.g. in an aqueous solution.

As used herein, the phrases “and mixtures thereof,” “and mixtures of two or more thereof,” “and a mixture thereof,” “and combinations thereof,” “and a combination thereof,” “or mixtures thereof,” “or a mixture thereof,” “or combinations thereof,” “or combinations of two or more thereof,” “or a combination thereof,” and the like are used interchangeably to denote that the listing of components immediately preceding the phrase, such as “A, B, C, D, or mixtures thereof” signify that the component(s) may be chosen from A, from B, from C, from D, from A+B, from A+B+C, from A+D, from A+C+D, etc., without limitation on the variations thereof. Thus, the components may be used individually or in any combination thereof.

For purposes of the present disclosure, it should be noted that to provide a more concise description, some of the quantitative expressions given herein are not qualified with the term “about.” It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value, unless otherwise stated.

All ranges and amounts given herein are intended to include sub-ranges and amounts using any disclosed point as an end point, and all endpoints are intended to be included unless expressly stated otherwise. Thus, a range of “1% to 10%, such as 2% to 8%, such as 3% to 5%,” is intended to encompass ranges of “1% to 8%,” “1% to 5%,” “2% to 10%,” and so on. All numbers, amounts, ranges, etc., are intended to be modified by the term “about,” whether or not expressly stated, unless expressly stated otherwise. Similarly, a range given of “about 1% to 10%” is intended to have the term “about” modifying both the 1% and the 10% endpoints. The term “about” is used herein to indicate a difference of up to +/−10% from the stated number, such as +/−9%, +/−8%, +/−7%, +/−6%, +/−5%, +/−4%, +/−3%, +/−2%, or +/−1%. Likewise, all endpoints of ranges are understood to be individually disclosed, such that, for example, a range of 1:2 to 2:1 is understood to disclose a ratio of both 1:2 and 2:1.

As used herein, if a component is described as being present “in an amount up to” a certain amount, it is intended that such component is, in fact, present in the composition, i.e. is present in an amount greater than 0%.

All amounts given herein are relative to the amount of active material, unless otherwise indicated.

All percentages, parts and ratios herein are based upon the total weight of the compositions of the present disclosure, unless otherwise indicated.

As used herein, the phrase “applying a composition onto hair” and variations thereof are intended to mean contacting the hair with at least one of the compositions of the disclosure, in any manner. It may also mean contacting the hair in an effective amount.

“Conditioning” as used herein means imparting to one or more hair fibers at least one property chosen from combability, moisture-retentivity, luster, shine, and/or softness. The state of conditioning can be evaluated by any means known in the art, such as, for example, measuring and comparing the ease of combability of the treated hair and of the untreated hair in terms of combing work (gm-in), and/or by consumer perception.

A “non-permanent” hair coloring composition or method as described herein is intended to mean a composition or method that does not employ oxidative hair dyes to alter the color of the hair.

The term “treat” (and its grammatical variations) as used herein refers to the application of the compositions of the present disclosure onto the surface of hair. The term “treat” (and its grammatical variations) as used herein also refers to contacting hair with the compositions of the present disclosure.

A “rinse-off” product refers to a composition such as a hair treatment composition that is removed from the hair after a processing time, e.g. is rinsed and/or washed with water. At least a portion, and typically most, of the composition is removed from the hair.

The term “stable” as used herein means that the composition does not exhibit greater than 10% change in hydrogen peroxide content and/or viscosity (measured at 25° C., 200 RPM for 30 seconds using a spindle M3 (Rheomat 180)) when stored at 25° C. for a specified period of time.

“Volatile,” as used herein, means having a flash point of less than about 100° C.

“Non-volatile,” as used herein, means having a flash point of greater than about 100° C.

As used herein, all ranges provided are meant to include every specific range within, and combination of sub ranges between, the given ranges. Thus, a range from 1-5, includes specifically 1, 2, 3, 4 and 5, as well as sub ranges such as 2-5, 3-5, 2-3, 2-4, 1-4, etc.

Unless otherwise defined for any specific embodiment, the term “substantially free” or “essentially free” as used herein means that there is less than about 5% by weight of a specific material added to a composition, based on the total weight of the composition. For example, the compositions may include less than about 4%, less than about 3%, less than about 2%, less than about 1.5%, less than about 1%, less than about 0.5%, less than about 0.1%, less than about 0.01%, less than about 0.001%, or less than about 0.0001% of the specified material. As such, it is contemplated that any component described herein for use in the hair treatment compositions can be present in the compositions in amounts less than about 5%, less than about 4%, less than about 3%, less than about 2%, less than about 1%, less than about 0.5%, less than about 0.1%, less than about 0.01%, less than about 0.001%, or less than about 0.0001%, and the composition will be considered “substantially free” of such material. A composition that is “free” of a component is understood to contain none of the specified component. However, it is understood that the terms “free” and “substantially free” refer to the amount of a component added to the composition, without including an amount of the component present in the composition as a minor component in a raw material. For example, a composition that is “free” of waxes may not have wax included as an intended component but may nevertheless contain a pigment that is coated with a wax, as such wax would be considered a minor component of the pigment material and would not be expected to provide benefits to the composition that would be expected by including a wax per se as an intended component.

Unless otherwise expressly stated, it is in no way intended that any method set forth herein be construed as requiring that its steps be performed in a specific order. Accordingly, where a method claim does not expressly recite an order to be followed by its steps or it is not specifically stated in the claims or descriptions that the steps are to be limited to a specific order, it not intended that any particular order be inferred.

All patents and publications are incorporated by reference. In the event of an inconsistency between the present disclosure and any patent or publications incorporated herein by reference, the present disclosure controls.

The examples that follow serve to illustrate embodiments of the present disclosure without, however, being limiting in nature. It will be apparent to those skilled in the art that various modifications and variations can be made in the compositions and methods of the invention without departing from the spirit or scope of the invention. Thus, it is intended that the present disclosure covers the modifications and variations that come within the scope of the appended claims and their equivalents.

### EXAMPLES

The following Examples are intended to be non-limiting and explanatory in nature only. In the Examples, amounts are expressed in percentage by weight (wt %) of active materials, relative to the total weight of the composition, unless otherwise indicated.

### Example 1—Hair Treatment Compositions

Hair treatment compositions 1A-1J according to the disclosure and comparative compositions C1 and 02 were prepared as shown in Tables 1-1 through 1-3. The pH of compositions 1A-1J and 02 were in the range of 3.5-4.5, and the pH of composition C1 was 9.3.

TABLE 1-1

| | 1A | 1B | 1C | 1D |
|---|---|---|---|---|
| AMINO ACIDS AND/OR | 0.27 | 0.27 | 0.27 | 0.27 |
| AMINO SULFONIC ACIDS | | | | |
| CETRIMONIUM CHLORIDE | 0.02 | 0.02 | 0.02 | 0.02 |
| LAURYL ALCOHOL | 0.35 | 0.37 | 0.35 | 0.52 |
| MYRISTYL ALCOHOL | 0.13 | 0.14 | 0.13 | 0.19 |
| PPG-2 BUTYL ETHER | 2.50 | 2.50 | 2.50 | 2.50 |
| CETYL ALCOHOL | 0.03 | 0.03 | 0.03 | 0.05 |
| SILICONES (DIMETHICONE, | 3.61 | 3.61 | 3.96 | 3.61 |
| AMODIMETHICONE, | | | | |
| PEG/PPG-4/12 DIMETHICONE) | | | | |
| GUAR HYDROXYPROPYL- | 0.18 | 0.23 | 0.15 | |
| TRIMONIUM CHLORIDE | | | | |
| DECETH-3 | | | | 2.30 |
| OLEYL ALCOHOL | 3.00 | 3.00 | 3.00 | 3.00 |
| COCAMIDE MIPA | 1.50 | 1.50 | 1.50 | 1.70 |
| COCO-BETAINE | 0.20 | 0.21 | 0.20 | 0.23 |
| LAURETH-2 | 2.20 | 2.30 | 2.20 | |
| SODIUM C14-16 OLEFIN | 2.00 | 2.20 | 2.00 | 2.70 |
| SULFONATE | | | | |
| PLANT OIL (APRICOT KERNEL | 1.01 | 1.01 | 1.01 | 1.01 |
| AND/OR SOYBEAN OIL) | | | | |
| TRIDECETH-3, TRIDECETH-6, | 0.18 | 0.18 | 0.20 | 0.18 |
| AND/OR TRIDECETH-10 | | | | |
| PPG-5-CETETH-10 PHOSPHATE | 0.27 | 0.24 | 0.27 | 0.30 |
| HYDROGEN PEROXIDE | 3.00 | 1.00 | 3.00 | 1.00 |
| SODIUM SULFITE | 0.25 | 0.25 | 0.25 | 0.25 |
| CARBOMER | 0.59 | 0.59 | 0.59 | 0.49 |
| PEG-100 STEARATE | 0.02 | 0.02 | 0.03 | 0.02 |
| STEARETH-6 | 0.02 | 0.02 | 0.02 | 0.02 |
| ADDITIVES (vitamins, | <3 | <3 | <3 | <3 |
| preservatives, hydrogen peroxide | | | | |
| stabilizers, pH adjusters, fragrance) | | | | |
| SOLVENTS (water and | QS | QS | QS | QS |
| non-aqueous solvents) | | | | |

TABLE 1-2

| | 1E | 1F | 1G | 1H |
|---|---|---|---|---|
| AMINO ACIDS AND/OR | 0.27 | 0.27 | 0.34 | 0.34 |
| AMINO SULFONIC ACIDS | | | | |
| CAPRYLYL GLYCOL | | | 0.60 | 0.60 |
| CETRIMONIUM CHLORIDE | 0.02 | 0.02 | | 0.03 |
| LAURYL ALCOHOL | 0.52 | 0.52 | | |
| MYRISTYL ALCOHOL | 0.19 | 0.19 | | |
| STEARYL ALCOHOL | | | 2.00 | 2.00 |
| HYDROXYPROPYL | | | 6.16 | 6.16 |
| STARCH PHOSPHATE | | | | |
| CETYL ESTERS | | | 1.00 | 1.00 |
| PPG-2 BUTYL ETHER | 2.50 | 2.50 | | |
| CETYL ALCOHOL | 0.05 | 0.05 | | |
| SILICONES (DIMETHICONE, | 3.60 | 3.60 | 0.80 | 1.46 |
| AMODIMETHICONE, | | | | |
| PEG/PPG-4/12 DIMETHICONE) | | | | |
| OLEYL ALCOHOL | 3.00 | 3.00 | | |
| COCAMIDE MIPA | 1.71 | 1.71 | | |
| COCO-BETAINE | 0.23 | 0.23 | | |
| LAURETH-2 | 2.56 | 2.56 | | |
| SODIUM C14-16 OLEFIN | 2.67 | 2.67 | | |
| SULFONATE | | | | |
| BEHENTRIMONIUM CHLORIDE | | | 1.58 | 1.58 |
| PLANT OIL (APRICOT KERNEL | 1.01 | 1.01 | 0.10 | 0.10 |
| AND/OR SOYBEAN OIL) | | | | |
| TRIDECETH-3, TRIDECETH-5, | 0.18 | 0.18 | 0.37 | 0.13 |
| TRIDECETH-6, AND/OR | | | | |
| TRIDECETH-10 | | | | |
| PPG-5-CETETH-10 PHOSPHATE | 0.27 | 0.27 | | |
| HYDROGEN PEROXIDE | 1.00 | 1.00 | 1.00 | 1.00 |
| SODIUM SULFITE | 0.25 | 0.25 | | |
| CANDELILLA WAX | | | 0.40 | 0.40 |
| CARBOMER | 0.49 | 0.59 | | |
| PEG-100 STEARATE | 0.02 | 0.02 | | |
| STEARETH-6 | 0.02 | 0.02 | | |
| POLYSORBATE 80 | 1.70 | 1.70 | | |
| QUATERNIUM-87 | | | 2.50 | 2.50 |
| ADDITIVES (vitamins, | <3 | <3 | <3 | <3 |
| preservatives, hydrogen peroxide | | | | |
| stabilizers, pH adjusters, fragrance) | | | | |
| SOLVENTS (water and | QS | QS | QS | QS |
| non-aqueous solvents) | | | | |

TABLE 1-3

| | 1I | 1J | C1 | C2 |
|---|---|---|---|---|
| AMINO ACIDS AND/OR | 0.27 | 0.27 | 0.50 | 0.27 |
| AMINO SULFONIC ACIDS | | | | |
| CETRIMONIUM CHLORIDE | 0.02 | 0.02 | | 0.02 |
| LAURYL ALCOHOL | 0.41 | 0.41 | 1.73 | 0.41 |
| MYRISTYL ALCOHOL | 0.15 | 0.15 | 0.63 | 0.15 |
| ETHANOLAMINE | | | 0.35 | |
| PPG-2 BUTYL ETHER | 2.50 | 2.50 | 5.00 | 2.50 |
| CETYL ALCOHOL | 0.04 | 0.04 | 0.15 | 0.04 |
| SILICONES (DIMETHICONE, | 3.61 | 3.61 | 1.50 | 3.61 |
| AMODIMETHICONE, | | | | |
| PEG/PPG-4/12 DIMETHICONE) | | | | |
| GUAR HYDROXYPROPYL- | 0.15 | 0.15 | | 0.15 |
| TRIMONIUM CHLORIDE | | | | |
| DECETH-3 | | | 8.10 | |
| OLEYL ALCOHOL | 3.00 | 3.00 | 6.00 | 3.00 |
| COCAMIDE MIPA | 1.71 | 1.71 | 6.50 | 1.71 |
| COCO-BETAINE | 0.23 | 0.23 | 0.75 | 0.23 |
| LAURETH-2 | 2.56 | 2.56 | | 2.56 |
| SODIUM C14-16 OLEFIN | 2.40 | 2.40 | 9.00 | 2.40 |
| SULFONATE | | | | |
| PLANT OIL (APRICOT KERNEL | 1.01 | 1.01 | | 1.01 |
| AND/OR SOYBEAN OIL) | | | | |
| TRIDECETH-3, TRIDECETH-6, | 0.18 | 0.18 | | 0.18 |
| AND/OR TRIDECETH-10 | | | | |
| PPG-5-CETETH-10 PHOSPHATE | 0.27 | 0.27 | 0.90 | 0.27 |
| HYDROGEN PEROXIDE | 1.00 | 3.00 | | |
| SODIUM SULFITE | 0.25 | 0.25 | 0.50 | 0.25 |
| CARBOMER | 0.50 | 0.50 | | 0.50 |
| PEG-100 STEARATE | 0.02 | 0.02 | | 0.02 |
| STEARETH-6 | 0.02 | 0.02 | | 0.02 |
| ADDITIVES (plant extracts, vitamins, | <2 | <2 | <2 | <2 |
| hydrogen peroxide stabilizers, | | | | |
| pH adjusters, fragrance, preservatives) | | | | |
| SOLVENTS (water and | QS | QS | QS | QS |
| non-aqueous solvents) | | | | |

### Example 2—Evaluation of Shine and Conditioning Performance

The following half-head studies were conducted by a panel of experts using volunteers having previously bleached and/or color-treated hair.

### Example 2A—Comparative Studies: Shine and Cosmetic Benefits

Three comparative studies were performed comparing a commercially-available shine treatment with hair treatment compositions according to the disclosure. First, all volunteers' hair was washed with shampoo, rinsed, and wrung dry. Next, composition C1, which is a commercially-available product for imparting shine to hair, was mixed with 6.7V aqueous hydrogen peroxide developer to provide a mixture (Clmix) containing 1% hydrogen peroxide and having a pH of about 6.5.

### Study Protocols

Study 1: On three volunteers, one-half of the head of hair was treated with composition 1A and the other half of the head of hair was treated with C1 mix by applying equal amounts of the respective compositions to each half head of hair, massaging through the hair to coat the hair, and leaving the compositions to process on the hair. The hair treated with composition 1A was rinsed after 5 minutes, while the hair treated with C1 mix was rinsed after 20 minutes as instructed by the product directions. The hair was then dried with a hair dryer.

Study 2: On three additional volunteers, one-half of the head of hair was treated with composition 1A and the other half of the head of hair was treated with C1ₘᵢₓ by applying equal amounts of the respective compositions to each half head of hair, massaging through the hair to coat the hair, and leaving the compositions to process on the hair. Each volunteers' hair was rinsed after 5 minutes. The hair was then dried with a hair dryer.

Study 3: On two additional volunteers, one-half of the head of hair was treated with composition 1E and the other half of the head of hair was treated with C1ₘᵢₓ by applying equal amounts of the respective compositions to each half head of hair, massaging through the hair to coat the hair, and leaving the compositions to process on the hair. The hair treated with composition 1E was rinsed after 5 minutes, while the hair treated with C1 ₘᵢₓ was rinsed after 20 minutes. The hair was then dried with a hair dryer.

### Results

The experts evaluated the properties and feel of the hair while wet, during the drying process, and once dried. The experts concluded that the hair treated with compositions 1A and 1E ranked on par with or better than hair treated with composition C1 ₘᵢₓ for properties of ease of wet detangling/combing, wet elasticity, dry combing, dry smoothness, dry shine, dry frizz control, and moisturized feel and reduced appearance of split ends on dry hair. This was seen both when composition C1 ₘᵢₓ was rinsed after 20 minutes, as instructed on the package (Study 1), or after 5 minutes (Study 2). It was noted, for example, that in Study 2, hair treated with composition 1A had noticeably better ease of combing and discipline with a sleeker and smoother appearance than hair treated for the same length of time with composition C1ₘᵢₓ.

Additionally, the experts observed that hair treated with compositions 1A and 1E ranked on par with or better than hair treated with composition C1ₘᵢₓ for shine, which was also observed both when composition C1ₘᵢₓ was rinsed after 20 minutes, as instructed on the package (Study 1), or after 5 minutes (Study 2). It was noted, for example, in Study 2, that hair treated with composition 1A had noticeably more shine (rated 5 out of 6) than hair treated for the same length of time with composition C1ₘᵢₓ (rated 2.5 out of 6).

### Example 2B—Additional Shine Study

An additional study was performed to evaluate the effectiveness of hair treatment compositions comprising hydrogen peroxide to impart shine to the hair. The tested compositions were identical other than the amounts of hydrogen peroxide present.

On two volunteers, one-half of the head of hair was treated with either composition 11 (1% hydrogen peroxide) or 1J (3% hydrogen peroxide), and the other half of the head of hair was treated with composition C2 (no hydrogen peroxide), by applying equal amounts of the respective compositions to each half head of hair, massaging through the hair to coat the hair, and leaving the compositions to process on the hair for 5 minutes before rinsing thoroughly. The hair was then dried with a hair dryer.

Once the hair was dry, the experts evaluated the hair for shine imparted. The experts concluded that the hair treated with composition 1J had the most shine followed by hair treated with composition 11, whereas hair treated with composition 02 demonstrated no appreciable shine relative to the hair before treatment.

The results of these studies demonstrate that hydrogen peroxide surprisingly imparts noticeable cosmetic benefits and shine to hair when hair is treated with compositions and methods according to the disclosure. Also surprisingly, the color of the hair was not lightened in these studies even with higher concentrations of hydrogen peroxide.

### Example 3—Stability Studies

In order to evaluate the stability of compositions according to the disclosure, the following studies were performed. Compositions 3A and 3B3 were prepared as shown in Table 3-1, and evaluated for viscosity and hydrogen peroxide content.

TABLE 3-1

| | 3A | 3B |
|---|---|---|
| AMINO ACIDS AND/OR | 0.25 | 0.25 |
| AMINO SULFONIC ACIDS | | |
| LAURYL ALCOHOL | 0.86 | 0.86 |
| MYRISTYL ALCOHOL | 0.31 | 0.31 |
| PPG-2 BUTYL ETHER | 2.50 | 2.50 |
| CETYL ALCOHOL | 0.08 | 0.08 |
| TETRASODIUM ETIDRONATE | | 0.06 |
| SODIUM SALICYLATE | | 0.04 |
| PEG/PPG-4/12 DIMETHICONE | 1.00 | 1.00 |
| DECETH-3 | 4.05 | 4.05 |
| OLEYL ALCOHOL | 3.00 | 3.00 |
| COCAMIDE MIPA | 3.25 | 3.25 |
| COCO-BETAINE | 0.38 | 0.38 |
| SODIUM C14-16 OLEFIN SULFONATE | 4.50 | 4.50 |
| PPG-5-CETETH-10 PHOSPHATE | 0.45 | 0.45 |
| HYDROGEN PEROXIDE | 1.00 | 1.00 |
| TETRASODIUM PYROPHOSPHATE | | 0.04 |
| SODIUM SULFITE | 0.25 | 0.25 |
| ERYTHORBIC ACID | 0.20 | 0.20 |
| POLOXAMER 184 | 0.60 | 0.60 |
| ADDITIVES (fragrance, preservatives, | <2 | <2 |
| pH adjusters, antioxidants) | | |
| SOLVENTS (water and non-aqueous solvents) | QS | QS |

Compositions 3A and 3B3 were identical other than the presence of hydrogen peroxide stabilizers in Composition 3B.

Each composition was divided into four samples in individual containers, which were stored sealed at temperatures of 400, 2500, 3700, or 4500. Each sample was evaluated by reassessing viscosity and hydrogen peroxide content at the time periods shown in Tables 3-2 through 3-3 below.

TABLE 3-2
Viscosity (mPa · s)*

| | Time | Temperature | | 3A | 3B |
|---|---|---|---|---|---|
| Initial | | | | 3147 | 3160 |
| | 1 | 4° | C. | 3315 | 3361 |
| | week | 25° | C. | 3302 | 3260 |
| | | 45° | C. | 3332 | 3332 |
| | 4 | 4° | C. | 3365 | 3332 |
| | weeks | 25° | C. | 3197 | 3260 |
| | | 45° | C. | 3017 | 2837 |
| | 8 | 4° | C. | 3386 | 3382 |
| | weeks | 25° | C. | 3315 | 3227 |
| | | 37° | C. | 2782 | 2715 |
| | | 45° | C. | 2954 | 2816 |

*measured at 200 RPM for 30 seconds using a spindle M3 (Rheomat 180)

TABLE 3-3
Hydrogen Peroxide Content (%)

| | Time | Temperature | | 3A | 3B |
|---|---|---|---|---|---|
| Initial | | | | 1.022 | 1.082 |
| | 1 | 4° | C. | 0.997 | 1.021 |
| | week | 25° | C. | 1.025 | 1.052 |
| | | 45° | C. | 0.984 | 1.033 |
| | 4 | 4° | C. | 0.997 | 1.042 |
| | weeks | 25° | C. | 1.012 | 1.049 |
| | | 45° | C. | 0.921 | 1.001 |
| | 8 | 4° | C. | 1.027 | 1.021 |
| | weeks | 25° | C. | 0.988 | 0.982 |
| | | 37° | C. | 0.958 | 0.973 |
| | | 45° | C. | 0.860 | 0.989 |

The stability studies in Tables 3-2 through 3-3 show that hair treatment compositions according to the disclosure are surprisingly stable over an extended period of time as demonstrated by what is considered minimal variation in viscosity and hydrogen peroxide content. The variations observed are considered to be within acceptable tolerances.

The results of the stability studies therefore demonstrate that hair treatment compositions containing silicone compounds, non-silicone fatty compounds, and a combination of anionic and nonionic surfactants surprisingly provides stability to the compositions despite the presence of hydrogen peroxide, even in the absence of hydrogen peroxide stabilizers.

### Example 4—Hair Treatment/Coloring Compositions

Compositions 4A-4E, which are hair treatment compositions according to the disclosure that include pigments and/or direct dyes to color the hair, were prepared as shown in Table 4-1.

TABLE 4-1

| | 4A | 4B | 4C | 4D | 4E |
|---|---|---|---|---|---|
| AMINO ACIDS | 0.27 | 0.27 | 0.25 | 0.27 | 0.27 |
| AND/OR AMINO | | | | | |
| SULFONIC ACIDS | | | | | |
| PEG/PPG-4/12 | 2.00 | 2.00 | 2.00 | 2.00 | 2.00 |
| DIMETHICONE | | | | | |
| AMODIMETHICONE | 1.50 | 1.50 | 1.50 | 1.50 | 1.50 |
| (and) TRIDECETH-6 | | | | | |
| (and) CETRIMONIUM | | | | | |
| CHLORIDE | | | | | |
| DIMETHICONE (and) | 1.50 | 1.50 | 1.50 | 1.50 | 1.50 |
| AMODIMETHICONE | | | | | |
| (and) TRIDECETH-10 | | | | | |
| (and) PEG-100 | | | | | |
| STEARATE (and) | | | | | |
| STEARETH-6 (and) | | | | | |
| TRIDECETH-3 | | | | | |
| PPG-2 BUTYL ETHER | 2.50 | 2.50 | 2.50 | 2.50 | 2.50 |
| GUAR | 0.18 | 0.18 | 0.18 | 0.18 | 0.18 |
| HYDROXYPROPYL- | | | | | |
| TRIMONIUM | | | | | |
| CHLORIDE | | | | | |
| OLEYL ALCOHOL | 3.00 | 3.00 | 3.00 | 3.00 | 3.00 |
| COCAMIDE MIPA | 1.45 | 1.45 | 1.45 | 1.45 | 1.45 |
| COCO-BETAINE | 0.65 | 0.65 | 0.65 | 0.65 | 0.65 |
| LAURETH-2 | 2.20 | 2.20 | 2.20 | 2.20 | 2.20 |
| SODIUM C14-16 | 5.00 | 5.00 | 5.00 | 5.00 | 5.00 |
| OLEFIN SULFONATE | | | | | |
| PLANT OIL (APRICOT | 1.01 | 1.01 | 1.00 | 1.01 | 1.01 |
| KERNEL AND/OR | | | | | |
| SOYBEAN OIL) | | | | | |
| LAURYL ALCOHOL | 0.50 | 0.50 | 0.50 | 0.50 | 0.50 |
| PPG-5-CETETH-10 | 0.27 | 0.27 | 0.27 | 0.27 | 0.27 |
| PHOSPHATE | | | | | |
| HYDROGEN | 3.00 | 3.00 | 3.00 | 3.00 | 3.00 |
| PEROXIDE | | | | | |
| SODIUM SULFITE | 0.25 | 0.25 | 0.25 | 0.25 | 0.25 |
| CARBOMER | 0.60 | 0.60 | 0.60 | 0.60 | 0.60 |
| 2-NITRO-5- | | 0.004 | | 0.005 | |
| GLYCERYL | | | | | |
| METHYLANILINE | | | | | |
| HC BLUE NO. 2 | | 0.063 | | 0.240 | |
| RED 28 | | | | | 0.048 |
| EXT. VIOLET 2 | | 0.100 | 0.500 | 0.330 | |
| HC BLUE NO. 15 | | 0.001 | | 0.002 | |
| BASIC VIOLET 2 | 0.003 | | | | |
| TETRABROMO- | | | | | 0.114 |
| PHENOL BLUE | | | | | |
| ADDITIVES (vitamins, | <2 | <2 | <2 | <2 | <2 |
| preservatives, hydrogen | | | | | |
| peroxide stabilizers, | | | | | |
| pH adjusters, fragrance) | | | | | |
| SOLVENTS (water and | QS | QS | QS | QS | QS |
| non-aqueous solvents) | | | | | |

Compositions 4A-4E can be used to provide advantageous benefits such as shine, softness, ease of combing/detangling, smoothness, reduced frizziness, etc., to the hair, while simultaneously providing non-permanent color to the hair.

Compositions 4F-4G can also be prepared using the components and amounts shown in Table 4-2.

TABLE 4-2

| | 4F | 4G |
|---|---|---|
| AMINO ACIDS AND/OR | 1.50 | 0.15 |
| AMINO SULFONIC ACIDS | | |
| AMODIMETHICONE | 4.50 | 1.00 |
| AND/OR DIMETHICONE | | |
| PPG-2 BUTYL ETHER | 2.50 | 2.50 |
| HYDROXYPROPYL | 0.50 | 1.00 |
| STARCH PHOSPHATE | | |
| MYRISTYL ALCOHOL | 2.00 | 4.00 |
| COCAMIDE MIPA | 2.50 | 1.00 |
| COCO-BETAINE | 1.00 | 0.25 |
| LAURETH-23 | 1.50 | 3.50 |
| SODIUM LAURYL | 1.00 | 3.50 |
| SULFOACETATE | | |
| PLANT OILS | 2.00 | 0.25 |
| LAURYL ALCOHOL | 1.50 | 3.50 |
| HYDROGEN PEROXIDE | 4.50 | 1.00 |
| SODIUM SULFITE | 0.25 | 0.25 |
| CARBOMER | 0.25 | 2.00 |
| RED 28 | | 0.008 |
| TETRABROMOPHENOL | 0.100 | 0.100 |
| BLUE | | |
| ADDITIVES (vitamins, | <2 | <2 |
| preservatives, hydrogen | | |
| peroxide stabilizers, | | |
| pH adjusters, fragrance) | | |
| SOLVENTS (water and | QS | QS |
| non-aqueous solvents) | | |

Compositions 4F-4G are also expected to provide advantageous benefits such as shine, softness, ease of combing/detangling, smoothness, reduced frizziness, etc., to the hair, while simultaneously providing non-permanent color to the hair.

In addition, compositions 4E, 4F, and 4G may be applied to hair that is alkaline in order to provide a blue or purple color to the hair.

The above data demonstrate that compositions according to the disclosure provide unexpected advantageous cosmetic benefits to the hair such as shine, softness, ease of combing/detangling, reduced frizziness, etc., and surprisingly require less processing time for such advantages to be achieved. In addition, the hair treatment compositions are surprisingly stable despite the presence of hydrogen peroxide.

1-52. (canceled)

53. A hair treatment composition comprising:
a. hydrogen peroxide and/or a hydrogen peroxide generating agent;
b. at least one surfactant chosen from anionic and/or nonionic surfactants;
c. at least one silicone compound;
d. at least one non-silicone fatty compound;
e. at least one polymer;
f. optionally at least one hydrogen peroxide stabilizer;
g. optionally at least one antioxidant;
h. optionally at least one pigment and/or direct dye; and
i. at least one solvent,
wherein the hair treatment composition has a pH less than about 7, and
wherein the hair treatment composition is stable for at least 24 hours.

54. The hair treatment composition according to claim 53, wherein the total amount of hydrogen peroxide and/or hydrogen peroxide generating agents ranges from about 0.5% to about 10% by weight, relative to the total weight of the composition.

55. The hair treatment composition according to claim 53, wherein the total amount of surfactants ranges from about 0.01% to about 15% by weight, relative to the total weight of the composition.

56. The hair treatment composition according to claim 53, wherein the hair treatment composition comprises:
at least one nonionic surfactant chosen from sorbitol esters of C8-C24 fatty acids or alkoxylated derivatives thereof, ethoxylated fatty alcohols, or fatty acid alkanolamides, and/or
at least one anionic surfactant chosen from sodium olefin sulfonates.

57. The hair treatment composition according to claim 53, further comprising at least one amphoteric surfactant and/or at least one cationic surfactant.

58. The hair treatment composition according to claim 53, wherein the total amount of silicone compounds ranges from about 0.1% to about 10% by weight, relative to the total weight of the composition.

59. The hair treatment composition according to claim 53, wherein the at least one silicone compound is chosen from dimethicone, amodimethicone, and/or derivatives thereof.

60. The hair treatment composition according to claim 53, wherein the total amount of non-silicone fatty compounds ranges from about 0.01% to about 10% by weight, relative to the total weight of the composition.

61. The hair treatment composition according to claim 53, comprising at least one plant oil.

62. The hair treatment composition according to claim 53, further comprising at least one polymer chosen from carbomers, quaternary ammonium polymers, and/or polysaccharide polymers.

63. The hair treatment composition according to claim 62, wherein the total amount of carbomers, quaternary ammonium polymers, and/or polysaccharide polymers ranges from about 0.01% to about 10% by weight, relative to the total weight of the composition.

64. The hair treatment composition according to claim 53, further comprising at least one amino acid and/or at least one amino sulfonic acid.

65. The hair treatment composition according to claim 64,
wherein the at least one amino acid is chosen from alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, valine, or taurine, or mixtures of two or more thereof,
wherein the at least one amino sulfonic acid is chosen from aminomethane sulfonic acid, aminopropane sulfonic acid, aminobutane sulfonic acid, aminohexane sulfonic acid, aminoisopropyl sulfonic acid, aminododecyl sulfonic acid, aminobenzene sulfonic acid, aminotoulene sulfonic acid, sulfanilic acid, chlorosulfanilic acid, diamino benzene sulfonic acid, amino phenol sulfonic acid, amino propyl benzene sulfonic acid, amino hexyl benzene sulfonic acid, or mixtures of two or more thereof.

66. The hair treatment composition according to claim 53, wherein the composition is free or substantially free of sulfate compounds and oxidative dye compounds.

67. The hair treatment composition according to claim 53, wherein the hair treatment composition has a pH ranging from about 3 to about 5.

68. The hair treatment composition according to claim 53, wherein the composition is free or substantially free of sulfate compounds and oxidative dye compounds.

69. A hair treatment composition comprising:
a. from at least 1.5% to about 6% of hydrogen peroxide;
b. at least one anionic surfactant;
c. at least one nonionic surfactant;
d. at least one silicone compound chosen from amodimethicone, dimethicone, and/or derivatives thereof, wherein the total amount of silicone compounds ranges from about 1% to about 6%
e. at least one plant oil, wherein the total amount of plant oils ranges from about 0.5% to about 2.5%;
f. at least one carbomer, wherein the total amount of carbomers ranges from about 0.01% to about 1.5%;
g. at least one additional component chosen from pigments and/or direct dyes, pH adjusters, amino acids and/or amino sulfonic acids, hydrogen peroxide stabilizers, antioxidants, vitamins, or mixtures of two or more thereof; and
h. water,
wherein the hair treatment composition has a pH ranging from about 3 to about 5, and
wherein all amounts are by weight, relative to the total weight of the hair treatment composition.

70. The hair treatment composition according to claim 69, wherein the composition comprises at least one pigment and/or at least one direct dye in a total amount ranging from about 0.001% to about 10% by weight, relative to the total weight of the composition.

71. The hair treatment composition according to claim 69, wherein the composition comprises at least one hydrogen peroxide stabilizer in a total amount ranging from about 0.001% to about 10% by weight, relative to the total weight of the composition.

72. A method of treating hair comprising:
(i) applying to the hair a composition comprising:
a. hydrogen peroxide and/or a hydrogen peroxide generating agent;
b. at least one surfactant chosen from anionic and/or nonionic surfactants;
c. at least one silicone compound;
d. at least one non-silicone fatty compound;
e. at least one polymer;
f. optionally at least one hydrogen peroxide stabilizer;
g. optionally at least one antioxidant;
h. optionally at least one pigment and/or direct dye; and
i. at least one solvent,
wherein the hair treatment composition has a pH less than about 7, and
wherein the hair treatment composition is stable for at least 24 hours,
(ii) leaving the composition on the hair for a leave-in period of time less than about 15 minutes, and
(iii) rinsing the hair,
wherein the method imparts softness, smoothness, ease of combing/detangling, frizz control, and/or reduced appearance of split ends to the hair.
